(19) United States
(12) Patent Application Publication
(10) Pub. No.: US 2022/0280584 A1
(43) Pub. Date: Sep. 8, 2022

(54) CROTON LECHLERI COMPOSITIONS FOR USE IN TREATING BLEEDING, WOUNDS AND INFECTIONS

(71) Applicant: ALPHYN BIOLOGICS, LLC, Cincinnati, OH (US)

(72) Inventors: Gary Michael PEKOE, Cincinnati, OH (US); Jazmyne Kristyne MINK, Cincinnati, OH (US); Steven Aaron PENTELNIK, Cincinnati, OH (US); Neal G. KOLLER, Annapolis, MD (US)

(21) Appl. No.: 17/631,861

(22) PCT Filed: Aug. 19, 2020
(86) PCT No.: PCT/US2020/047082

§ 371 Date: Jan. 31, 2022

Related U.S. Application Data
(60) Provisional application No. 62/888,829, filed on Aug. 19, 2019.

(51) Int. Cl. A61K 36/47; A61L 15/40; A61P 17/02

(52) U.S. Cl. CPC A61K 36/47; A61L 15/40; A61P 17/02

(57) ABSTRACT

The present disclosure provides for the treatment of wounds, treatment of chronic wounds, treatment of traumatic wounds, treatment of acute wounds, treating or preventing or reducing the risk of a bacterial infection of wounds, inducing blood clotting of a wound, promotion of blood coagulation, promotion of clot formation, promotion of blood clotting by accelerating the normal platelet clotting pathway, promotion of wound healing, promotion of wound occlusion, promotion of wound occlusion thereby reducing scarring, promotion of tissue regeneration, promotion of tissue growth, promotion of tissue growth thereby accelerating wound healing, or a combination thereof, in a subject via the administration of a treated bandage comprising a therapeutically effective amount of an extract of the Croton lechleri tree.

### CROSS-REFERENCE TO RELATED APPLICATIONS

This application claims the benefit of U.S. Provisional Application No. 62/888,829 filed Aug. 19, 2019. The disclosure of the application is incorporated herein by reference.

### SUMMARY

The present invention is generally related to the treatment of bleeding (or excessive bleeding) wounds and/or infections via the topical administration of a pharmaceutical compositions comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard. The concentration of components and performance standards of latex of Croton lechleri, preferably the concentration of components and performance standards of filtered latex of Croton lechleri, preferably the concentration of components and performance standards of filtered latex of Croton lechleri Müll.Arg of the reference standard are found in Tables 1a-e.

### BRIEF DESCRIPTION OF THE DRAWINGS

FIG. 1 depicts a representative Total Ion Chromatogram as well as additional Multiple Reaction Monitoring spectra that identify the marker compounds in an AB-101 composition.

FIG. 2A depicts the NMR spectra of 3 lots of AB-101 in D₂O—the top spectra is for Lot 00, the middle spectra is for Lot 01, and the bottom spectra is for Lot 02.

FIG. 2B depicts the overlay of the NMR spectra of Lots 00, 01, and 02 of AB-101 in D₂O.

FIG. 3A depicts the Nuclear Magnetic Resonance (NMR) spectra of 3 lots of AB-101 in d₄-Methanol—the top spectra is for Lot 00, the middle spectra is for Lot 01, and the bottom spectra is for Lot 02.

FIG. 3B depicts the overlay of the NMR spectra of Lots 00, 01, and 02 of AB-101 in d₄-Methanol.

FIG. 4A depicts the NMR spectra of 4 lots of AB-101 in d₄-Methanol—the top spectra is for Lot 00, the upper middle spectra is for Lot 01, the lower middle is for Lot 02, and the bottom spectra is for Lot X.

FIG. 4B depicts the overlay of the NMR spectra of Lots 00, 01, 02, and X of AB-101 in d₄-Methanol.

FIG. 5 depicts bar graphs comparing the AB-101 lot analysis results for A) gallocatechin B) epigallocatechin C) catechin D) epicatechin and E) taspine.

FIG. 6 depicts a representative Total Ion Chromatogram of dimethylcedrusin.

FIG. 7 depicts Case 1: AB-101 wound treatment top left (thigh).

FIG. 8 depicts Case 2: AB-101 wound treatment inside left leg (thigh).

FIG. 9 depicts Case 3: AB-101 wound treatment inside right top foot.

### DEFINITIONS

Before the present compositions and methods are described, it is to be understood that this invention is not limited to the particular processes, compositions, or methodologies described, as these may vary. It is also to be understood that the terminology used in the description is for the purpose of describing the particular versions or embodiments only and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

As used herein, the terms below have the meanings indicated.

It must also be noted that as used herein and in the appended claims, the singular forms “a,” “an,” and “the” include plural reference unless the context clearly dictates otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50% means in the range of 45%-55%.

As used herein, the term “AB-101” maybe used interchangeably with latex of Croton lechleri, preferably filtered latex of Croton lechleri, preferably filtered latex of Croton lechleri Müll.Arg., and botanical raw material. The latex is excreted material from the wounded trunk of Croton lechleri, preferably of Croton lechleri Müll.Arg. In all such instances the latex is the whole latex. In all such instances, the latex is unfractionated.

“Administering” when used in conjunction with a therapeutic, such as AB-101, means to administer a therapeutic directly into or onto a target tissue or to administer a therapeutic to a patient whereby the therapeutic positively impacts the tissue to which it is targeted. Thus, as used herein, the term “administering”, when used in conjunction with a composition of embodiments herein, can include, but is not limited to, providing the composition into or onto the target tissue; providing the composition to a patient by, e.g., topical application whereby the therapeutic reaches the target tissue. “Administering” a composition may be accomplished topically or in combination with other known techniques.

As used herein the term “cellulitis/erysipelas” is defined as a diffuse bacterial skin infection characterized by spreading areas of redness, edema, and/or induration.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the pharmaceutical composition, composition or the method includes only the elements, steps, or ingredients specifically recited in the particular claimed embodiment or claim.

As used herein, the term “consisting essentially of” or “consists essentially of” means that the pharmaceutical composition, or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the composition or method that treats the specified condition (e.g., nutrient depletion) is the specifically recited therapeutic(s) in the particular embodiment or claim.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “excipient” and “pharmaceutically acceptable excipient” as used herein are intended to be generally synonymous, and is used interchangeably with, the terms “carrier,” “pharmaceutically acceptable carrier,” “diluent,” “pharmaceutically acceptable diluent.”

As used herein, the term “extract” refers to the liquid that runs between the bark and the wood portions of the tree, which is and remains unfractionated.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

As used in each of the embodiments here in, sap may be include among others sap, latex, resin, extract, or any combination of the foregoing.

As used herein, the term “therapeutic” or “therapeutic agent” or “pharmaceutically active agent” means an agent utilized to treat, combat, ameliorate, prevent or improve an unwanted condition or disease of a patient.

In part, embodiments of the present invention are directed to the treatment of, but not limited to, Streptococcus pyogenes infection, Staphylococcus aureus infection, methicillin-resistant Staphylococcus aureus (MRSA) infection, Mupirocin-resistant MRSA, Enterococcus faecalis infection, Gram-positive bacterial infection, Gram-negative bacteria infection, cellulitis/erysipelas, wound infection, burn infection, major cutaneous abscesses, impetigo, Mupirocin-resistant impetigo, Vancomycin resistant bacteria infection, Mupirocin resistant bacteria infection, Clostridium difficile infection, drug-resistant Neisseria gonorrhoeae infection, Streptococcus pneumoniae infection, drug-resistant Streptococcus pneumoniae infection, drug-resistant Klebsiella pneumoniae infection, drug-resistant Malaria infection, Multi-drug resistant (MDR) infection, Extensively drug-resistant (XDR) Tuberculosis infection, Escherichia coli (E. coli) infection, Shiga toxin-producing Escherichia coli (E. coli) infection, infections caused by bacteria possessing Enzyme NDM-1 (New Delhi Metallo-beta-lactamase-1), Clostridium difficile infection, Enterococcus infection, Mycobacterium tuberculosis infection, Mycoplasma genitalium infection, Streptococcus infection, Campylobacter infection, Neisseria gonorrhoeae infection, Gamma proteobacteria infection, Enterobacteriaceae infection, Carbapenem-Resistant Enterobacteriaceae, infection, Klebsiella pneumoniae infection, Salmonella infection, E. coli infection, Pseudomonadales infection, Acinetobacter infection, Pseudomonas aeruginosa infection, MDR Pseudomonas aeruginosa infection, and Coagulase-negative Staphylococcus infection.

The term “therapeutically acceptable” refers to those compositions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid.

The phrase “therapeutically effective” is intended to qualify the amount of active ingredients used in the treatment of a disease or disorder or on the effecting of a clinical endpoint.

A “therapeutically effective amount” or “effective amount” of a composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per application will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of the composition of this invention is typically an amount such that when it is administered in a physiologically tolerable excipient composition, it is sufficient to achieve an effective systemic concentration or local concentration in the tissue.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The phrase “treated bandage” refers to a bandage that has either pharmaceutical compositions of the present invention or a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, that is either first applied to or impregnated into a bandage.

The term “topical” includes administering to any skin or mucosal surface or being suitable for such administration. In some embodiments, “topical” may be the skin surface. Skin surface includes any part of the body, including but not limited to face, hands, legs, neck, abdominal area, eyes, nose, and chest. Mucosal surface includes, without limitation, mucosa of the mouth or oral mucosa, lips, tongue, nasal, buccal mucosa, palate, gingiva, nasopharynx, respiratory epithelium, conjunctiva, vagina, cervix, and urethral mucosa.

As used herein, the term “wound” is defined as an injury to living tissue caused by a cut, blow, or other impact, typically one in which the skin is cut or broken.

As used herein the term “wound infection” is defined as a bacterial infection characterized by purulent drainage from a wound with surrounding redness, edema, and/or induration.

Also provided are embodiments wherein any embodiment herein may be combined with any one or more of the other embodiments, unless otherwise stated and provided the combination is not mutually exclusive.

The chemical defenses of plants include complex mixtures of organic compounds and typically do not involve individual substances; these compounds appear in different concentrations (majority or minority) within various products derived from natural species. The biological activities of these products can be found to originate from their ability to interact among themselves and other substances through synergistic, additive, antagonistic effects—and can be optimized through the modification of the pharmacokinetics and/or pharmacodynamics of the component substances. The biological effects may occur from the interaction with all the organic compounds or by the interaction among certain components, which may present themselves as majority or minority. Accordingly, when described herein, AB101 consists of the whole latex obtained from the Croton lechleri tree—it is unfractionated—but is selected based upon the presence of select components that meet the reference standard as described herein.

In natural product research, the major compound's role and mechanism in its associated biological activity is commonly investigated. Thus, in the scientific literature, there are innumerous published studies where the major components have been found to be responsible for these activities. However, this disregards the possible interactions among the totality of compounds that may be present at lower concentrations in natural products. A study carried out with the essential oil Thymus vulgaris and its major constituent, thymol identified that the effect of the constituents of the oil was not phytotoxic to lettuce seeds, whereas the isolated action of thymol caused significant inhibitory effects on seed germination, raising the possibility of a partial inhibition of thymol activity by other components of the oil. Demonstrating the importance of considering the interactions among all components of the product.

For example, evaluation of the activity of the sap of Dracaena cochinchinensis and three active constituents regarding the analgesic activity from the inhibition of currents on the TRPV1 channel, induced by capsaicin. As a result, the authors found that the combination of the three active components of the sap is responsible for the analgesic activity of the species in question, where these components act synergistically, as the compounds found in greater concentration were not directly responsible for the biological activity found.

Another consideration regarding interactions among the active components of a natural product is the ability to alter the pharmacokinetics of the components when compared with the administration of these molecules in isolation. This can be achieved by modifying the absorption, distribution, metabolism and elimination profiles. A study reported the pharmacokinetic profile of chlorogenic acid and coryloin alone in comparison with the product formed by the hydroalcoholic extract of Pharbitis nil and Corydalis tuber, DA-9701, which contains the two components in equivalent concentrations. Results showed a significant increase in the AUC of coryloin when DA-9701 was administered compared with the two compounds in isolation, both orally. This increase in AUC can be explained by decreased hepatic and/or gastrointestinal first-pass metabolism compared with pure coryloin. In addition, there may be inhibition of corticosteroid presystemic metabolism by other components of DA-9701.

Another example is the complexity of metabolic pathways and the complexity of essential oils, extracts and herbal products may be directly related to the recorded biological effect. In a study with essential oil of Eucalyptus tereticornis and its major constituents, it was observed that all three major constituents reinforce the constricting effect of acetylcholine in the trachea of rats, however with a stimulus of potassium, the essential oil presents a relaxing effect, may be due to the inhibition of acetylcholinesterase activity.

Croton lechleri (a member of the family Euphorbiaceae, commonly called the spurge family) has approximately 1,300 species of plants that are either herbaceous (plants that have no persistent woody stem above ground, shrub (a woody plant which is smaller than a tree and has several main stems arising at or near the ground), tree (a perennial plant with an elongated stem, or trunk, supporting branches and leaves in most species), or liana (any of various long-stemmed, woody vines that are rooted in the soil at ground level and use trees, as well as other means of vertical support, to climb up to the canopy to get access to well-lit areas of the forest) forms. The Croton genus is a diverse and complex group of flowering plants ranging from herbs and shrubs to trees. The Croton genus is widely distributed in tropical and subtropical regions around the world.

Dragon's blood refers to a bright red resin that is obtained from different species of a number of distinct plant genus: Croton, Dracaena, Daemonorops, Calamus rotang and Pterocarpus. The red resin has been in continuous use since ancient times as varnish, medicine, incense, and dye. The name dragon's blood is used to refer to all of the above plant genus, often without any distinction as to the genus or species it is coming from. Those with the same genus will be similar in any therapeutic or nutritional value, with factors such as local soil, local rainfall, local humidity, local sunlight, local fauna and the like imparting variability and inconsistency. However, the difference between the red resin coming from Croton versus Daemonorops (a genus of rattan palms in the family Arecaceae found primarily in the tropics and subtropics of southeastern Asia with a few species extending into southern China and the Himalayas) will be significant. The Croton and Daemonorops genus originate from opposite sides of the world so their components are different and therefore specificity of source plant is important to deliver the desired medicinal benefits or avoid undesirable toxic results. For example, milky white latex that is often toxic or at least irritating to the skin is common to the members of the spurge or Euphorbiaceae family. Therefore selecting the specific genus, species, and local geographical area of the spurge or Euphorbiaceae family is essential to having the possibility for the latex to have specific and repetitive medicinal properties.

A handful of Croton species found in the South America rainforest (in countries of Bolivia, Brazil, Colombia, Ecuador and Peru) Central America and Mexico produce the red latex, commonly known as dragon's blood, that has medicinal properties. The dragon's blood trees grown in these areas include Croton lechleri, Croton draco, Croton palanostigma, Croton sordidus, Croton urucurana, and Croton xalapenesis.

In certain embodiments, the specific dragon's blood tree of the present application is Croton lechleri Müll.Arg. of the Family: Euphorbiaceae. Dragon's blood is also referred to as Sangre de drago (Peru), Sangre de grado (Ecuador).

While the desired medicinal properties could be found by extracting the compositions from either the leaves or bark, in preferred embodiments, it is the deep red latex of Croton lechleri, preferably filtered latex of Croton lechleri, preferably filtered latex of Croton lechleri Müll.Arg, wherein the composition contains at least the concentration of components of latex of Croton lechleri, preferably the concentration of components of filtered latex of Croton lechleri, preferably the concentration of components of filtered latex of Croton lechleri Müll.Arg of the reference standard, that is also referred to as latex, that is utilized. According to Langenheim (2003) resin “is a lipid-soluble mixture of volatile and non-volatile terpenoid and/or phenolic secondary compounds that are usually secreted in specialized structures located either internally or on the surface of the plant and are of potential significance in ecological interactions”. By contrast, latex, is a mixture of terpenoids, phenolic compounds, acids, carbohydrates, etc. having a protective role (Lewisohn 1991) and produced in special cells called laticifers (Fahn 1979). Chemical characterization of dragon's blood is species specific and has been undertaken by many authors. For example, it is possible to distinguish between dragon's blood from some individual species used in works of art, since it has been sold as a colorant for many centuries (Baumer and Dietemann 2010). Dragon's blood of Croton spp. is usually referred to as latex due to the fact that it is secreted and stored by laticifers, and its major constituents are polymeric anthocyanidins, which co-occur with many minor constituents, including diterpenes and simple phenols (Salatino et al. 2007). Dragon's blood secreted by stems of Pterocarpus officinalis is also called latex (Weaver 1997; Guerrero and Guzman 2004); however, information about the chemical composition of the exudate and its ecological function is poorly known. Dragon's blood derived from species of Dracaena and Daemonorops is a phenolic resin (Langenheim 2003), with well-recognized chemical content (e.g. Gonzalez et al. 2000; Shen et al. 2007; Sousa et al. 2008). Sometimes, dragon's blood is referred to as latex (e.g. Philipson 2001). However, this could prove to be a source of confusion, since plants produce other exudates referred to by that name, such as xylem latex and phloem latex, which are entirely different in terms of their location, chemical composition and function. The resin is obtained through tapping the tree or other common draining methods. Draining the tree latex has the additional benefit of not having to use complex and costly extraction technology to obtain the desired composition from either the leaves or bark. The latex of Croton lechleri Müll.Arg. of the present application is then filtered in a 30 micron filter to remove plant debris and thick, resinous material. Chemical characterization of dragon's blood is local geography specific and has not been undertaken by prior authors.

Medicinal and toxic properties of various species of the Croton genus have been ascribed to a wide variety of chemical compounds, such as terpenoids and steroids, alkaloids, and phenolic compounds, the latter including predominantly flavonoids, lignans, and proanthocyanidins. Some embodiments of the present application utilize the whole latex, thereby leveraging the “organic” synergy of all the latex components as intended by nature. The molecular classes found in latex of Croton lechleri Müll.Arg. of the present application which provide the desired medicinal benefits of Croton lechleri Müll.Arg. are: Alkaloids, Diterpenes, Lignans, Phenols, Phytosterols, Proanthocyanidins, Sterols and Tannins.

For a pharmaceutical composition to be effective in treating topical wounds, this composition needs to have properties that including but not limited to: antibacterial performance to fight infection, to stop bleeding the composition needs to be effective at blood clotting, blood coagulating and providing hemostatic properties, to promote healing the composition needs to increase cell proliferation, stimulate fibroblasts migration and have antioxidant properties and to provide wound protection, the composition needs to have the ability to form a film over the wound. In addition, the composition needs to have a safety profile for use in topical applications where the composition has low systemic blood absorption (i.e. passage into the blood stream) and where the composition that is absorbed has a low partition coefficient as measured by LogP. The LogP represents the concentration of solute in the organic and aqueous partition. A low LogP means a higher partition or concentration in the aqueous solute. This is desirable from a safety standpoint. A higher LogP indicates the composition is more likely to absorbed and retained in the body via organs and tissues, while lower LogP indicates higher safety via the composition would be natural eliminated, not absorbed or retained that could lead to build up of toxic compounds.

AB-101 uses the unique composition of the entire latex of the Croton lechleri Müll. Arg. The novelty of this invention is identifying the pharmaceutical AB-101 composition that has all the performance properties listed above to treat topical bleeding wounds, fight infections and promote healing with the appropriate safety profile. This represents a complex multivariant solution that optimizes multiple performance properties where the solution is not obvious to one familiar with the art.

In the embodiments disclosed herein, the latex of Croton lechleri, preferably filtered latex of Croton lechleri, preferably filtered latex of Croton lechleri Müll.Arg, that is utilized is not fractionated, but does contain at least the concentration of certain components and performance standards of the reference standard as set forth in Tables 1a-e.

Some embodiments herein are directed to a method of identifying a composition of latex of Croton lechleri, preferably a composition of filtered latex of Croton lechleri, preferably a composition of filtered latex of Croton lechleri Müll.Arg comprising: (a) determining the concentration of components of latex of Croton lechleri, preferably the concentration of components of filtered latex of Croton lechleri, preferably the concentration of components of filtered latex of Croton lechleri Müll.Arg,; (b) comparing the concentrations of the components to the concentrations of the components of a reference standard; and (c) identifying a composition of latex of Croton lechleri, preferably a composition of filtered latex of Croton lechleri, preferably a composition of filtered latex of Croton lechleri Müll.Arg, wherein the composition contains at least the concentration of components of latex of Croton lechleri, preferably the concentration of components of filtered latex of Croton lechleri, preferably the concentration of components of filtered latex of Croton lechleri Müll.Arg of the reference standard.

Some embodiments herein are directed to a method of identifying a composition of latex of Croton lechleri, preferably a composition of filtered latex of Croton lechleri, preferably a composition of filtered latex of Croton lechleri Müll.Arg for use in treating wounds in a subject comprising: (a) determining the concentration of components of latex of Croton lechleri, preferably the concentration of components of filtered latex of Croton lechleri, preferably the concentration of components of filtered latex of Croton lechleri Mëll.Arg,; (b) comparing the concentrations of the components to the concentrations of the components of a reference standard; and (c) identifying a composition of latex of Croton lechleri, preferably a composition of filtered latex of Croton lechleri, preferably a composition of filtered latex of Croton lechleri Müll.Arg for use in treating wounds in a subject, wherein the composition contains at least the concentration of components of latex of Croton lechleri, preferably the concentration of components of filtered latex of Croton lechleri, preferably the concentration of components of filtered latex of Croton lechleri Müll.Arg of the reference standard.

Some embodiments herein are directed to a method of identifying a composition of latex of Croton lechleri, preferably a composition of filtered latex of Croton lechleri, preferably a composition of filtered latex of Croton lechleri Müll.Arg for use in treating or preventing or reducing the risk of a bacterial infection of wounds in a subject comprising: (a) determining the concentration of components of latex of Croton lechleri, preferably the concentration of components of filtered latex of Croton lechleri, preferably the concentration of components of filtered latex of Croton lechleri Müll.Arg,; (b) comparing the concentrations of the components to the concentrations of the components of a reference standard; and (c) identifying a composition of latex of Croton lechleri, preferably a composition of filtered latex of Croton lechleri, preferably a composition of filtered latex of Croton lechleri Müll.Arg for use in treating or preventing or reducing the risk of a bacterial infection of wounds in a subject, wherein the composition contains at least the concentration of components of latex of Croton lechleri, preferably the concentration of components of filtered latex of Croton lechleri, preferably the concentration of components of filtered latex of Croton lechleri Müll.Arg of the reference standard.

Some embodiments herein are directed to a method of identifying a composition of latex of Croton lechleri, preferably a composition of filtered latex of Croton lechleri, preferably a composition of filtered latex of Croton lechleri Müll.Arg for use in inducing blood clotting of a wound in a subject comprising: (a) determining the concentration of components of latex of Croton lechleri, preferably the concentration of components of filtered latex of Croton lechleri, preferably the concentration of components of filtered latex of Croton lechleri Müll.Arg,; (b) comparing the concentrations of the components to the concentrations of the components of a reference standard; and (c) identifying a composition of latex of Croton lechleri, preferably a composition of filtered latex of Croton lechleri, preferably a composition of filtered latex of Croton lechleri Müll.Arg for use in blood clotting of a wound in a subject, wherein the composition contains at least the concentration of components of latex of Croton lechleri, preferably the concentration of components of filtered latex of Croton lechleri, preferably the concentration of components of filtered latex of Croton lechleri Müll.Arg of the reference standard.

Embodiments of the present invention are directed to pharmaceutical compositions of latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, of which have been found to be useful in the successful treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

In some embodiments the pharmaceutical compositions are administered topically. Embodiments are directed to pharmaceutical compositions comprising latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, disclosed herein together with a pharmaceutically acceptable carrier, as well as methods of making and using the compounds and compositions.

Certain embodiments are directed to methods selected from the treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

Other embodiments are directed to methods selected from the treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof, in need of such treatment, comprising administering to said patient a therapeutically effective amount of a composition, wherein the composition contains at least the concentration of components of latex of Croton lechleri, preferably the concentration of components of filtered latex of Croton lechleri, preferably the concentration of components of filtered latex of Croton lechleri Müll.Arg of the reference standard, according to the present invention.

Also provided is the use of certain extracts of Croton lechleri disclosed herein in the manufacture of a medicament for the treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

### Pharmaceutical Compositions

Embodiments herein are directed to pharmaceutical compositions comprising latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, which have been found to be useful in the successful treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

The pharmaceutical AB-101 composition uses the whole Croton lechleri Mëll. Arg latex. The art is full of examples where the Croton lechleri Müll. Arg latex is fractionated, individual components are isolated or individual components are minimized or eliminated. This may be a result of the specific use. For the pharmaceutical grade of AB-101, the preference is to use the entire extract to leverage the synergy associated with all the active compounds.

Importantly, the variation of all the compounds vary greatly as indicated by the geography, environment, soil, elevation and age of the trees to name a few of the key variables. These variations are highlighted by Thiago Vaz Lopes, Dragon's blood (Croton lechleri Müll. Arg; An Update on the Chemical Composition and Medical Applications of This Natural Plant Extract, Revista Brasileira de Higiene and Animal Health (v.7, n2) p. 167-192 (2013)).

The AB-101 novelty is based upon identifying the linkage between the specific compounds and their levels of concentration within AB-101 via a bioassay to in vitro efficacy and confirming via human use testing as an effective treatment for wound treating, bleeding treatment, and fighting infections. The utility of this novel discovery is the basis for developing a pharmaceutical drug and a medicinal product that will meet the FDA standards.

The FDA has established the requirement of having a bioassay that correlates the performance of the botanical raw material based on the chemical characterization of the composition and changes therein, to the efficacy against wound treating, bleeding treatment, and fighting infections.

The Croton lechleri Müll. Arg latex latex is complex, difficult and not straightforward to define since its composition uses the full accompaniment of all of the bioactive materials comprising the Croton lechleri Müll. Arg latex. Net, finding the critical active markers and performance and safety tests requires novel discovery.

The FDA requires the identification of the critical biomarkers or active constituents that drives the bioactivity. To that end, the critical biomarkers and their associated concentrations for AB-101 have never been published, defined or identified as associated with wound healing properties, antimicrobial activity and safety for treatment of wound treating, bleeding treatment, and fighting infections. Without this information, the FDA will not grant a drug status for medicinal use which is at the heart of becoming a pharmaceutical drug.

“Pharmaceutical Products” means any product, compound, me or therapeutic which is subject to regulation as a drug, medicine or controlled substance by a foreign equivalent of the United States Food and Drug Administration.

FDA guidance on botanicals states:

Because of the heterogeneous nature of a botanical drug and possible uncertainty about its active constituents, one of the critical issues for botanical drugs is ensuring that the therapeutic effect for marketed drug product batches is consistent. In general, therapeutic consistency can be supported by a “totality of the evidence” approach, including the following considerations:
Botanical raw material control (e.g, agricultural practice and collection).
Quality control by chemical test(s) (e.g., analytical tests such as spectroscopic and/or chromatographic methods that capture the active chemical constituents of a botanical drug substance) and manufacturing control (e.g., process validation).
Biological assay (e.g. a biological assay that reflects the drug's known or intended mechanism of action) and clinical data (for details regarding use of clinical data in ensuring therapeutic consistency.

By using the whole Croton lechleri Müll. Arg latex a unique synergy can be obtained across the entire composition that meets the specific bioassay performance targets. Table 1 shows 16 bioactives found in the pharmaceutical grade of AB-101. These bioactive compounds provide efficacy for: antimicrobial, antiviral, anti-inflammatory, cell proliferation to promote healing, anticancer, hemostatic, antioxidant and fibroblast stimulation to promote healing. By maintaining the Croton lechleri Müll. Arg latex intact, a tremendous synergy is obtained across wound healing and preventing infections.

Table A shows bioactive compounds found in the whole Croton lechleri Müll. Arg latex of AB-101 and their properties

TABLE A

| | | | | | Cell | | | | Stimulating |
|---|---|---|---|---|---|---|---|---|---|
| Bioactive | Phytochemical | | | | Proliferative | | | | fibroblasts |
| Chemical | Compound | Anti- | | Anti- | (Wound | Anti- | | Anti- | (Wound |
| Components | Class | microbial | Antiviral | inflammatory | Healing) | cancer | Hemostat | oxidant | Healing) |
| 1,3,5- | Flavonoid | X | | | | | | | |
| Trimethyoxybenzene | | | | | | | | | |
| 2,4,6- | Phenol | X | X | | | X | | | |
| Trimethoxyphenol | | | | | | | | | |
| 3',4-O-dimethylcedrusin | Lignin | | | | | | | | X |
| 4-O- | Lignin | | | | X | | | | |
| Dimethylcedrusin | | | | | | | | | |
| Boldine, iso | Alkaloid | | | X | | X | | | |
| Catechin | Flavonoid | X | X | X | | | X | X | |
| Epicatechin | Flavonoid | | X | X | | | | X | |
| Epigallocatechin | Phenol | X | | | | | | X | |
| Flavan-3-ols | Flavonoids | X | X | | | | | | |
| Gallocatechin | Flavonoid | X | X | | X | | | X | |
| Magnoflorine | Alkaloid | X | X | X | | | | | |
| Proanthocyanidins | Polyphenols | X | X | X | X | X | | X | |
| Procyanidin | Flavonoids | X | X | X | X | | | | |
| Prodelphinidin | Tannins | X | | | | | | | |
| Sitosterol-Beta- | Phytosterol | X | X | X | | | | | |
| Glucopyranoside | | | | | | | | | |
| Taspine | Alkaloid | X | X | X | | X | | | |

Some embodiments herein are directed to a pharmaceutical composition comprising latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, of embodiments herein and a pharmaceutically acceptable excipient. Optionally, the pharmaceutical composition may further comprise one or more other therapeutic ingredients. In embodiments, the pharmaceutical composition comprises a therapeutically effective amount of the latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard. In embodiments, the pharmaceutical composition is suitable for topical administration or is a topical pharmaceutical composition.

Embodiments herein are directed to pharmaceutical compositions comprising latex of Croton lechleri, preferably filtered latex of Croton lechleri, preferably filtered latex of Croton lechleri Müll.Arg., wherein the pharmaceutical composition does not contain a pharmaceutically acceptable excipient. In certain embodiments, latex of Croton lechleri, preferably filtered latex of Croton lechleri, preferably filtered latex of Croton lechleri Müll.Arg. comprises one or more compounds selected from: gallocatechin, epigallocatechin, catechin, epicatechin, taspine, and dimethylcedrusin and combinations thereof. Each of gallocatechin, epigallocatechin, catechin, epicatechin, taspine, and dimethylcedrusin may be present in the latex of Croton lechleri, preferably filtered latex of Croton lechleri, preferably filtered latex of Croton lechleri Müll.Arg. in at least the amounts found in Table 1a or any combination of such amounts.

Embodiments herein are directed to pharmaceutical compositions comprising latex of Croton lechleri, preferably filtered latex of Croton lechleri, preferably filtered latex of Croton lechleri Müll.Arg. and a pharmaceutically acceptable excipient. In certain embodiments, latex of Croton lechleri, preferably filtered latex of Croton lechleri, preferably filtered latex of Croton lechleri Müll.Arg. comprises one or more compounds selected from: gallocatechin, epigallocatechin, catechin, epicatechin, taspine, and dimethylcedrusin and combinations thereof. Each of gallocatechin, epigallocatechin, catechin, epicatechin, taspine, and dimethylcedrusin may be present in the latex of Croton lechleri, preferably filtered latex of Croton lechleri, preferably filtered latex of Croton lechleri Müll.Arg. in at least the amounts found in Table 1a or any combination of such amounts.

TABLE 1a

| | Exemplary Amount present in the |
|---|---|
| Compound | latex (PPM is in μg/g) |
| Gallocatechin | at least about 110 PPM |
| Epigallocatechin | at least about 780 PPM |
| Catechin | at least about 1.6 PPM |
| Epicatechin | at least about 2 PPM |
| Taspine | at least about 45 PPM |
| Dimethylcedrusin | at least about 0.1 PPM |

TABLE 1b

| | Exemplary Amount present in the latex as |
|---|---|
| Compound or compounds | a % of total Proanthocyanidins (PAC) |
| Gallocatechin and | at least about 60% |
| Epigallocatechin combined | |
| Epigallocatechin | at least about 45% |

TABLE 1c
Exemplary Antibiotic Activity

| Bacteria | Exemplary MIC | Exemplary MBC |
|---|---|---|
| Methicillin-susceptible | 50 μg/mL or less | 50 μg/mL or less |
| Staphylococcus | | |
| aureus (MSSA) | | |
| Methicillin-resistant | 50 μg/mL or less | 50 μg/mL or less |
| Staphylococcus | | |
| aureus (MRSA) | | |
| Pseudomonas aeruginosa | 50 μg/mL or less | 50 μg/mL or less |
| Streptococcus pyongenes | 50 μg/mL or less | 50 μg/mL or less |

TABLE 1d
Exemplary LogP for each of gallocatechin, epigallocatechin,
catechin, epicatechin, and taspine

| | |
|---|---|
| LogP: Skin Permeation | at least about 2.5 |
| LogP Flux Calculation | at least about less than 550 μg/cm²/hr |

TABLE 1e
Exemplary Additional Properties

| | |
|---|---|
| Film Forming Properties | Present as observed on skin |

If the latex of Croton lechleri, preferably filtered latex of Croton lechleri, preferably filtered latex of Croton lechleri Müll.Arg. fails to contain the amounts of gallocatechin, epigallocatechin, catechin, epicatechin, taspine, and dimethylcedrusin in at least the amounts set forth in Table 1a, it is not suitable for use in the pharmaceutical compositions and methods of use described herein.

In some embodiments, the gallocatechin is in an amount of at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, at least about 200 mg, or a range between any two of these values.

In some embodiments, the epigallocatechin is in an amount of at least about 780 mg, at least about 790 mg, at least about 800 mg, at least about 810 mg, at least about 820 mg, at least about 830 mg, at least about 840 mg, at least about 850 mg, at least about 860 mg, at least about 870 mg, at least about 880 mg, at least about 890 mg, at least about 900 mg, at least about 910 mg, at least about 920 mg, at least about 930 mg, at least about 940 mg, at least about 950 mg, at least about 960 mg, at least about 970 mg, at least about 980 mg, at least about 990 mg, at least about 1000 mg, at least about 1010 mg, at least about 1020 mg, at least about 1030 mg, at least about 1040 mg, at least about 1050 mg, at least about 1060 mg, at least about 1070 mg, at least about 1080 mg, at least about 1090 mg, at least about 1100 mg, at least about 1110 mg, at least about 1120 mg, at least about 1130 mg, at least about 1140 mg, at least about 1150 mg, at least about 1160 mg, at least about 1170 mg, at least about 1180 mg, at least about 1190 mg, at least about 1200 mg, at least about 1210 mg, at least about 1220 mg, at least about 1230 mg, at least about 1240 mg, at least about 1250 mg, at least about 1260 mg, at least about 1270 mg, at least about 1280 mg, at least about 1290 mg, at least about 1300 mg, at least about 1310 mg, at least about 1320 mg, at least about 1330 mg, at least about 1340 mg, at least about 1350 mg, at least about 1360 mg, at least about 1370 mg, at least about 1380 mg, at least about 1390 mg, at least about 1400 mg, at least about 1410 mg, at least about 1420 mg, at least about 1430 mg, at least about 1440 mg, at least about 1450 mg, at least about 1460 mg, at least about 1470 mg, at least about 1480 mg, at least about 1490 mg, at least about 1500 mg, at least about 1510 mg, at least about 1520 mg, at least about 1530 mg, at least about 1540 mg, at least about 1550 mg, at least about 1560 mg, at least about 1570 mg, at least about 1580 mg, at least about 1590 mg, at least about 1600 mg, at least about 1610 mg, at least about 1620 mg, at least about 1630 mg, at least about 1640 mg, at least about 1650 mg, at least about 1660 mg, at least about 1670 mg, at least about 1680 mg, at least about 1690 mg, at least about 1700 mg, or a range between any two of these values.

In some embodiments, the catechin is in an amount of at least about 1.6 mg, at least about 1.7 mg, at least about 1.8 mg, at least about 1.9 mg, at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, at least about 4.5 mg, at least about 4.6 mg, at least about 4.7 mg, at least about 4.8 mg, at least about 4.9 mg, at least about 5.0 mg, at least about 5.1 mg, at least about 5.2 mg, at least about 5.3 mg, at least about 5.4 mg, at least about 5.5 mg, at least about 5.6 mg, at least about 5.7 mg, at least about 5.8 mg, at least about 5.9 mg, at least about 6.0 mg, at least about 6.1 mg, at least about 6.2 mg, at least about 6.3 mg, at least about 6.4 mg, at least about 6.5 mg, at least about 6.6 mg, at least about 6.7 mg, at least about 6.8 mg, at least about 6.9 mg, at least about 7.0 mg, at least about 7.1 mg, at least about 7.2 mg, at least about 7.3 mg, at least about 7.4 mg, at least about 7.5 mg, at least about 7.6 mg, at least about 7.7 mg, at least about 7.8 mg, at least about 7.9 mg, at least about 8.0 mg, at least about 8.1 mg, at least about 8.2 mg, at least about 8.3 mg, at least about 8.4 mg, at least about 8.5 mg, at least about 8.6 mg, at least about 8.7 mg, at least about 8.8 mg, at least about 8.9 mg, at least about 9.0 mg, at least about 9.1 mg, at least about 9.2 mg, at least about 9.3 mg, at least about 9.4 mg, at least about 9.5 mg, at least about 9.6 mg, at least about 9.7 mg, at least about 9.8 mg, at least about 9.9 mg, at least about 10.0 mg, at least about 10.1 mg, at least about 10.2 mg, at least about 10.3 mg, at least about 10.4 mg, at least about 10.5 mg, at least about 10.6 mg, at least about 10.7 mg, at least about 10.8 mg, at least about 10.9 mg, at least about 11.0 mg, or a range between any two of these values.

In some embodiments, the epicatechin is in an amount of at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, at least about 4.5 mg, at least about 4.6 mg, at least about 4.7 mg, at least about 4.8 mg, at least about 4.9 mg, at least about 5.0 mg, at least about 5.1 mg, at least about 5.2 mg, at least about 5.3 mg, at least about 5.4 mg, at least about 5.5 mg, at least about 5.6 mg, at least about 5.7 mg, at least about 5.8 mg, at least about 5.9 mg, at least about 6.0 mg, at least about 6.1 mg, at least about 6.2 mg, at least about 6.3 mg, at least about 6.4 mg, at least about 6.5 mg, at least about 6.6 mg, at least about 6.7 mg, at least about 6.8 mg, at least about 6.9 mg, at least about 7.0 mg, at least about 7.1 mg, at least about 7.2 mg, at least about 7.3 mg, at least about 7.4 mg, at least about 7.5 mg, at least about 7.6 mg, at least about 7.7 mg, at least about 7.8 mg, at least about 7.9 mg, at least about 8.0 mg, at least about 8.1 mg, at least about 8.2 mg, at least about 8.3 mg, at least about 8.4 mg, at least about 8.5 mg, at least about 8.6 mg, at least about 8.7 mg, at least about 8.8 mg, at least about 8.9 mg, at least about 9.0 mg, at least about 9.1 mg, at least about 9.2 mg, at least about 9.3 mg, at least about 9.4 mg, at least about 9.5 mg, at least about 9.6 mg, at least about 9.7 mg, at least about 9.8 mg, at least about 9.9 mg, at least about 10.0 mg, or a range between any two of these values.

In some embodiments, the taspine is in an amount of 45 mg, at least about 46 mg, at least about 47 mg, at least about 48 mg, at least about 49 mg, at least about 50 mg, at least about 51 mg, at least about 52 mg, at least about 53 mg, at least about 54 mg, at least about 55 mg, at least about 56 mg, at least about 57 mg, at least about 58 mg, at least about 59 mg, at least about 60 mg, at least about 61 mg, at least about 62 mg, at least about 63 mg, at least about 64 mg, at least about 65 mg, or a range between any two of these values.

In some embodiments, the dimethylcedrusin is in an amount of 0.1 mg, at least about 0.11 mg, at least about 0.12 mg, at least about 0.13 mg, at least about 0.14 mg, at least about 0.15 mg, at least about 0.16 mg, at least about 0.17 mg, at least about 0.18 mg, at least about 0.18 mg, at least about 0.19 mg, at least about 0.20 mg, at least about 0.21 mg, at least about 0.22 mg, at least about 0.23 mg, at least about 0.24 mg, at least about 0.25 mg, at least about 0.26 mg, at least about 0.27 mg, at least about 0.28 mg, at least about 0.29 mg, at least about 0.30 mg, at least about 0.31 mg, at least about 0.32 mg, at least about 0.33 mg, at least about 0.34 mg or a range between any two of these values.

The pharmaceutical composition of AB-101 as described and claimed herein is a plant sourced material that meets the criteria of being consistently reproducible between batch to batch and reliably delivers the desired health benefits via topical application that may be used in a pharmaceutical composition. It can be used to treat Acute Bacterial Skin or Skin Structure Infections, treat wounds, reduce the risk of bacterial infections and increase or promote blood clotting. Plant sourced materials face the challenge that changes in environmental weather, climate, rainfall, time of harvest (via season, time of day or month), changes in geography, longitude location, latitude location, altitude, changes in soil condition, harvesting protocols and many additional conditions can alter the characteristics of the plant that could impact quality. This can impact the plant's bioactivity resulting in inconsistency in achieving desired performance outcome. This creates a challenge in defining a pharmaceutical grade of dragon's blood to deliver consistent and reproducible therapeutic benefits. This is further compounded by the wide variety of the different species called dragon's blood. For example, phytochemical and anti-staphylococcal biofilm assessment of Dracaena draco L. Spp .draco resin, referred as dragon's blood, is “inactive in the maximum tested concentration of 1000 mcg/ml against free living staphylococci.” In contrast, AB-101 (latex of Croton lechleri, preferably filtered latex of Croton lechleri, preferably filtered latex of Croton lechleri Müll.Arg. with the appropriate levels of gallocatechin, epigallocatechin, catechin, epicatechin, taspine, and dimethylcedrusin) is effective against Staphylococcus specifically methicillin-susceptible Staphylococcus aureus (MSSA) or the shorten nomenclature staph bacteria and in particular methicillin-resistant Staphylococcus aureus (MRSA) and in particular Mupirocin resistant MRSA. The generic name of the Croton lechleri resin, ie, dragon's blood, or Sangre de grado, creates confusion in defining a plant-derived pharmaceutical and demonstrates that not all Croton lechleri plants are the same, nor do they provide similar benefits.

The benefits of AB-101, filtered or unfiltered latex of Croton lechleri, preferably filtered latex of Croton lechleri, preferably filtered latex of Croton lechleri Müll.Arg., is its ability to deliver consistent results for treating the pathogens between batch to batch in spite of all the confounding conditions. The challenge in using the whole latex is to identify the compounds that deliver performance based on the many bio-active compounds comprising the latex. Even within the same species, grown in a similar location, there are variations in chemical content and bioactivity of the whole latex that unexpectedly varies in its ability to fight and kill pathogens.

Methodology that can identify the whole latex is effective by having an assay that determines when a batch meets the predetermined performance criteria. Having a unique analytical and microbiological assay enables the ability to identify which batch of filtered or unfiltered latex of Croton lechleri, preferably filtered latex of Croton lechleri, preferably filtered latex of Croton lechleri Müll.Arg, has the combination of components that will consistently deliver the desired outcome.

AB-101 botanical raw material (BRM) is a complex botanical product that is a latex of Croton lechleri, preferably filtered latex of Croton lechleri, preferably filtered latex of Croton lechleri Müll.Arg. that contains certain marker compounds (catechin, gallocatechin, epicatechin, epigallocatechin, taspine, and dimethylcedrusin) in specified amounts (see Table 1a). Utilization of liquid chromatography with tandem mass spectrometry (LC-MS/MS) can be used to characterize the existence and levels of such marker compounds for batch to batch consistency and repeatable performance of AB-101. Marker compounds in AB-101 BRM include the proanthocyanidins: catechin, gallocatechin, epicatechin, and epigallocatechin, the alkaloid taspine, as well as dimethylcedrusin.

The published and accepted taxonomic classification of Croton lechleri Müll.Arg. is the following (van Ee & Berry, 2011, Riina et al, 2009, The Plant List, 2012, The Angiosperm Phylogeny Group, 2009):

### Division: Streptophyta

### Class: Equisetopsida

### Subclass: Magnoliidae

### Order: Malpighiales

### Family: Euphorbiaceae

### Genus: Croton

### Subgenus Adenophylli

### Section: Cyclostigma

### Subsection: Cyclostigma

Species: Croton lechleri Müll.Arg.

Biodiversity of botanicals plays a major role in constituent chemical compound characterization. Chemical compounds utilized for as important batch to batch consistency of AB-101 need to 1) demonstrate antimicrobial or cicatrizant properties, 2) be present in AB-101, and 3) be detectable using analytical techniques. Using these criteria, the analytical efforts focused on 3 classes of compounds: polyphenols (proanthocyanidins), alkaloids (taspine), and lignin (dimethylcedrusin). Within the proanthocyanidin class, 4 specific compounds were focused on: catechin, epicatechin, gallocatechin, and epigallocatechin. The compound of importance within the alkaloid class is taspine. Finally, the compound of importance within the lignin class is dimethylcedrusin. Each of these compounds fulfills the three required elements detailed above. The following are the chemical structures of the 6 compounds utilized as important markers for batch to batch consistency of AB-101.

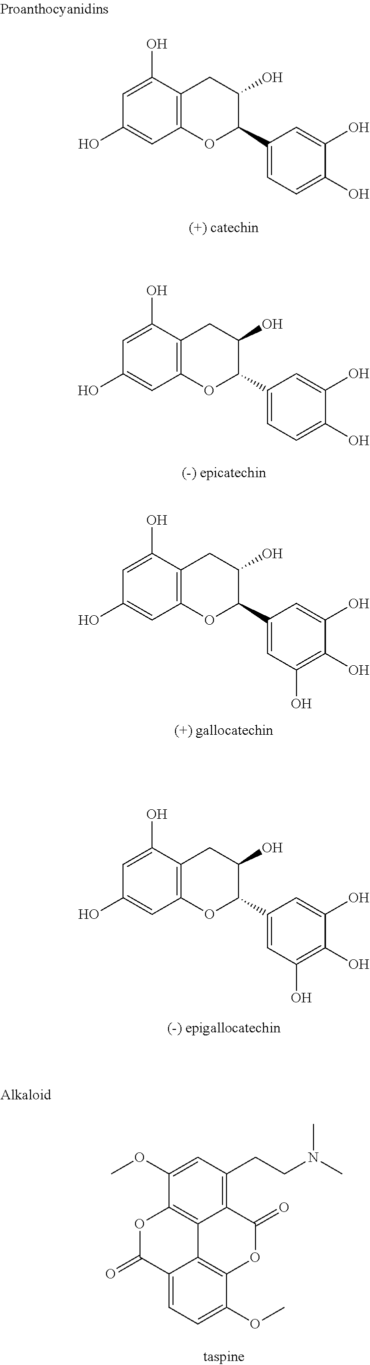 [SMILES: Oc1cc(O)c2c(c1)O[C@H](c1ccc(O)c(O)c1)[C@@H](O)C2 ; Oc1cc(O)c2c(c1)O[C@@H](c1ccc(O)c(O)c1)[C@H](O)C2 ; Oc1cc(O)c2c(c1)O[C@H](c1cc(O)c(O)c(O)c1)[C@@H](O)C2 ; Oc1cc(O)c2c(c1)O[C@@H](c1cc(O)c(O)c(O)c1)[C@H](O)C2 ; COc1ccc2c(=O)oc3c(OC)cc(CCN(C)C)c4c(=O)oc1c2c34]

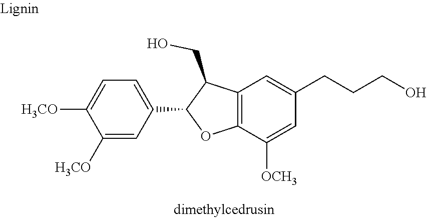 [SMILES: COc1ccc([C@H]2Oc3c(OC)cc(CCCO)cc3[C@@H]2CO)cc1OC]

For characterization studies, AB-101 extract was lyophilized and the lyophilized powder was subjected to three different extraction methods.

Method 1—Ultrasonic polyphenol extraction. The lyophilized AB-101 extract was dissolved into methanol. The resultant emulsion was then subjected to sonication for 10 minutes followed by centrifugation to remove particulates for 5 minutes. The supernatant was then subjected to LC-MS/MS analysis.

Method 2—Soxhlet extraction. The lyophilized AB-101 extract was subjected to a Soxhlet extraction with 80% ethanol. The ethanol was removed via a rotary evaporator. The resultant material was then subjected re-suspended in ethanol then subjected to LC-MS/MS analysis.

Method 3—Polyphenol extraction. The lyophilized AB-101 extract was incubated with methanol overnight at room temperature and in the dark. The supernatant was then filtered using Whatman filters, dried, and then re-suspended in methanol. The resultant material was then subjected to LC-MS/MS analysis.

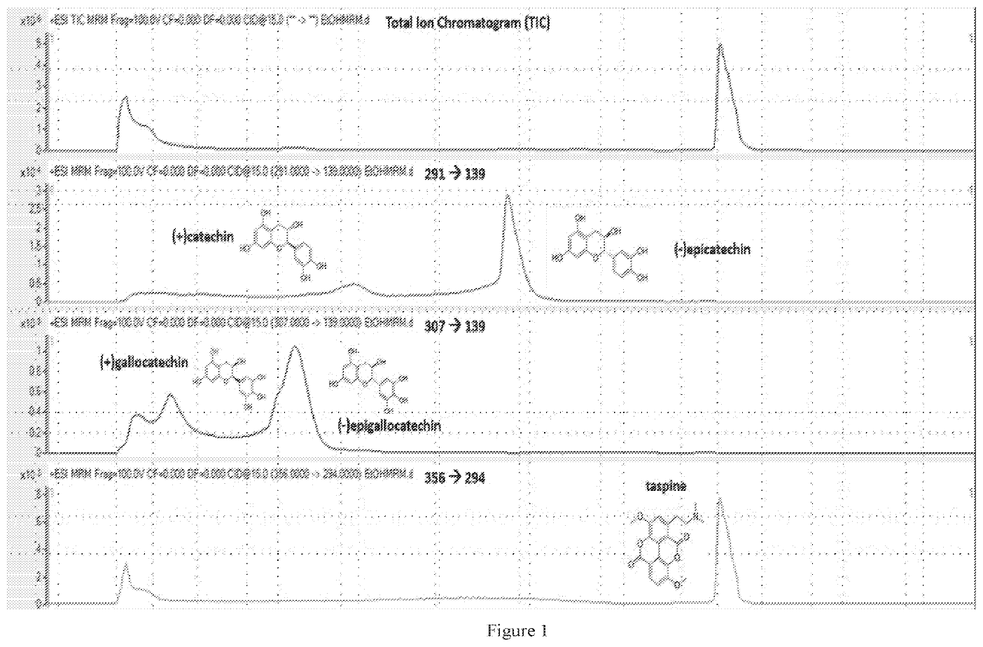

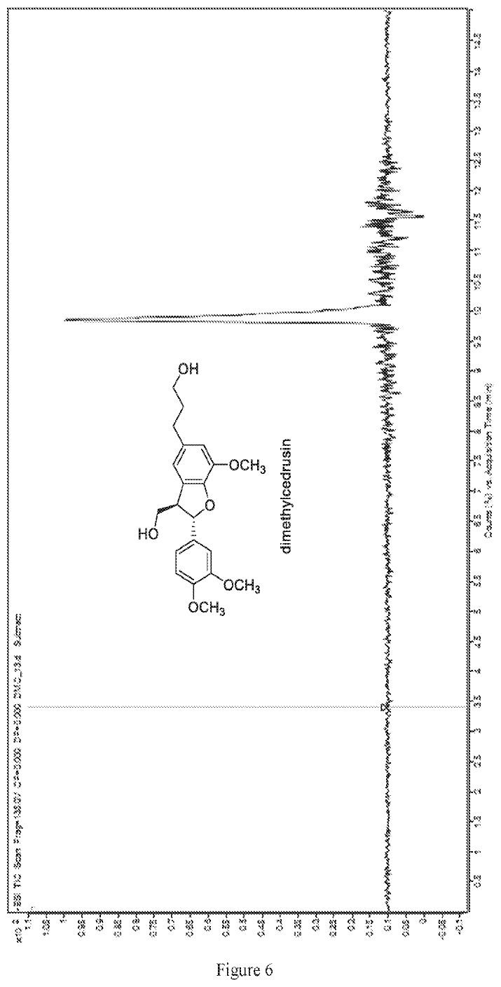

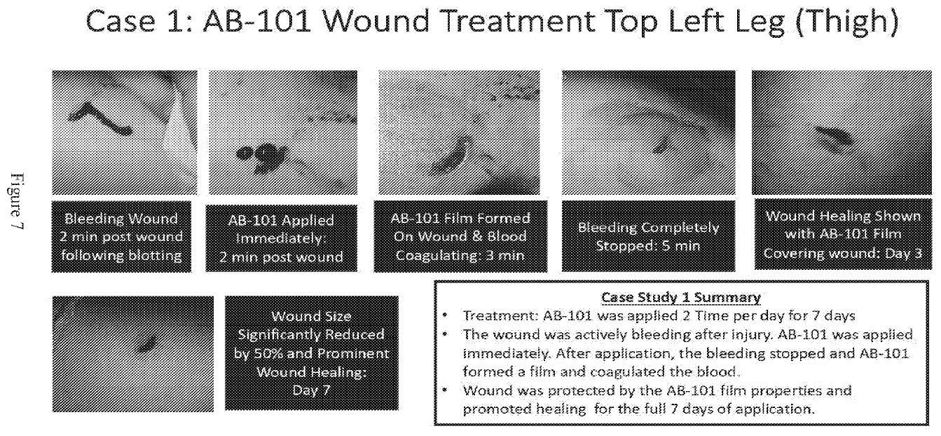

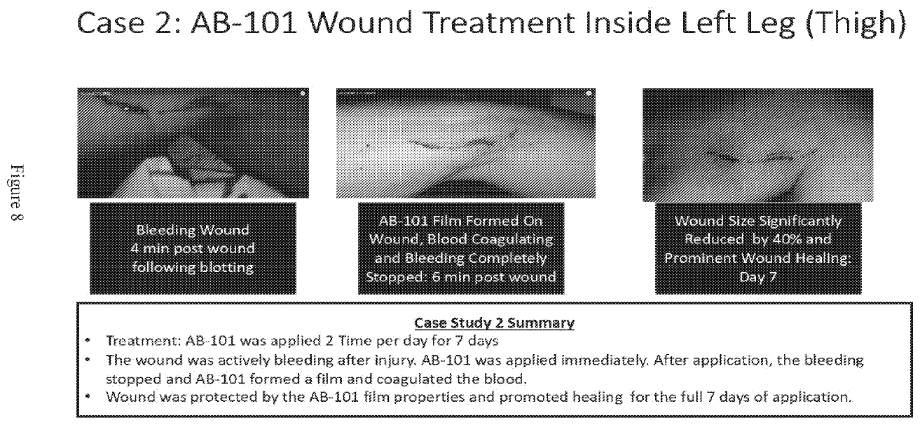

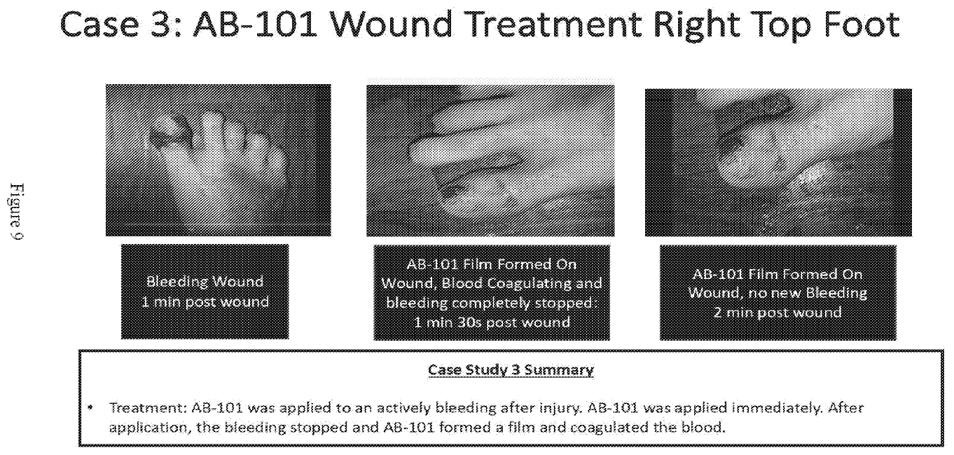

FIG. 1 depicts a representative Total Ion Chromatogram as well as additional Multiple Reaction Monitoring spectra that identify the important marker compounds in an AB-101 extract. While FIG. 6 depicts a representative Total Ion Chromatogram of dimethylcedrusin. The compounds are detectable using any of the three extraction methods.

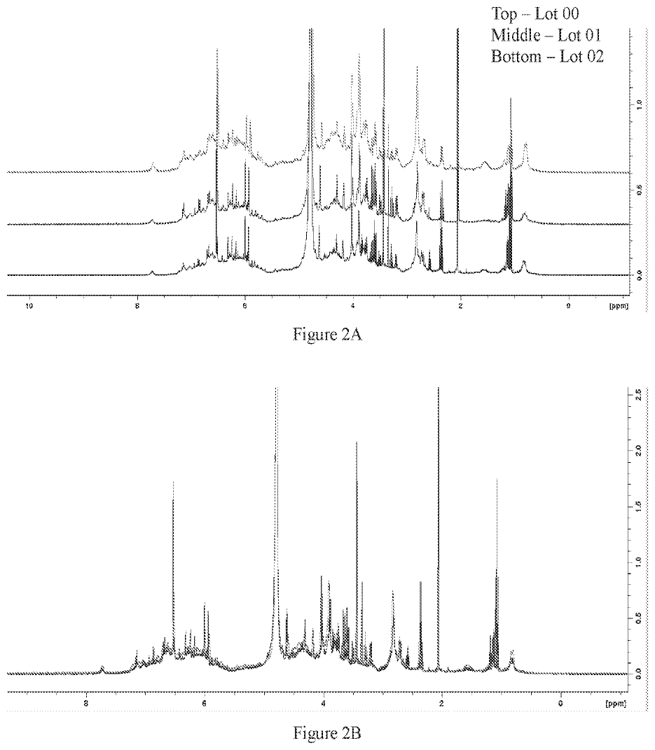

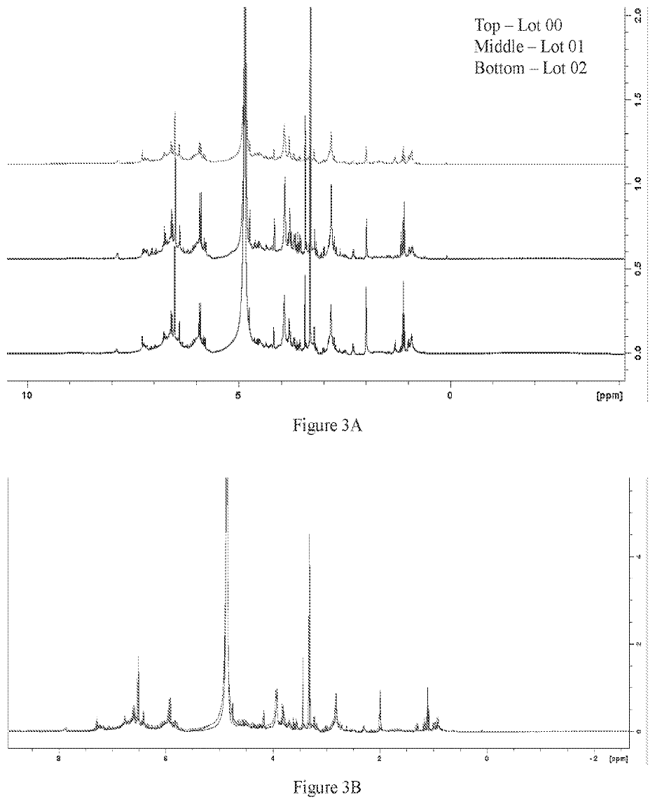

Biodiversity contributes to vast amounts of variability. In order to capture this variability, an NMR method utilizing a “spectral fingerprint” was used with an overlapping a reference standard. These fingerprinting captures most components within AB-101 and would be quantifiable using Nuclear Magnetic Resonance (NMR). Examples of NMR spectra using three different AB-101 lots (Lots 00, 01, and 02 respectively) and two different deuterated solvents (D₂O and d₄-Methanol respectively) are shown in FIGS. 2A and 3A with overlays of each solvents spectra being shown in FIGS. 2B and 3B and demonstrated no significant variability.

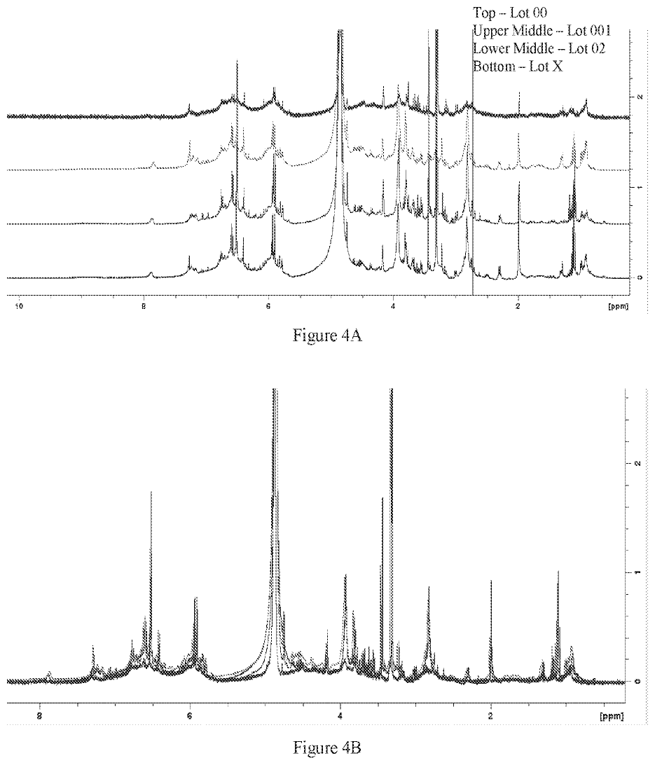

In another NMR analysis using the d₄-Methanol as the solvent, 4 distinct lots of AB-101 (Lots 00, 01, 02, and X respectively) are compared. NMR spectra of each lot are shown in FIG. 4A with overlays of each lots spectra being shown in FIG. 4B. While the fingerprint of the 4 lots looks similar, there are important differences. This is shown by comparing the concentration level in ppm based on LC-MS/MS Quantification and qualitative NMR “fingerprinting” on the marker compounds of catechin, epicatechin, gallocatechin, epigallocatechin, and taspine. The results are shown in Table 2 and indicate that lots 1 and 2 are more similar and lots X and 0 have the largest differences.

TABLE 2

AB-101 Lots Characterization
PPM (μg/g)

| Lot | X | 00 | 01 | 02 |
|---|---|---|---|---|
| Gallocatechin (GC) | 164.2 | 91.9 | 135.0 | 139.9 |
| Epigallocatechin (EGC) | 1357.6 | 380.7 | 1219.5 | 996.3 |
| Catechin (C) | 2.0 | 6.7 | 8.8 | 8.2 |
| Epicatechin (EC) | 2.6 | 5.2 | 8.3 | 6.1 |
| Taspine (T) | 50.4 | 43.4 | 50.1 | 51.1 |
| Dimethylcedrusin | 0.1 | 0.1 | 0.1 | 0.1 |

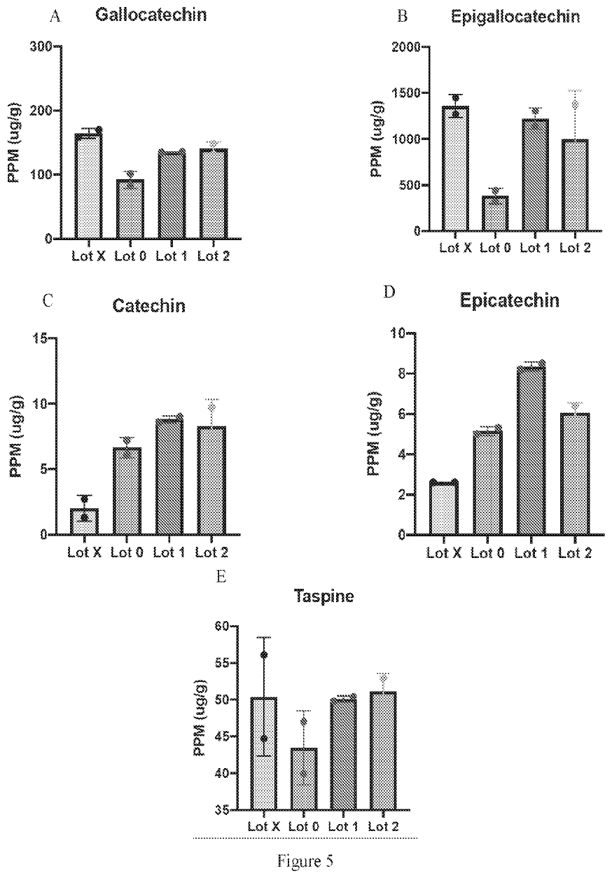

FIG. 5A-E depicts bar graphs comparing the AB-101 lot analysis results for each of the 5 marker compounds.

Lot 00 is an example of a lot that is not suitable for use in the pharmaceutical compositions and the methods of use described herein. Lots X, 01 and 02 are exmaples of lots that are suitable for use in the pharmaceutical compositions and the methods of use described herein.

Zheng-Ping Chen publication (Studies on the Anti-Tumour, Anti-Bacterial and Wound-Healing Properties of Dragon's Blood, Planta Med. 60 (1994)) demonstrates the non-obviousness of identifying the optimum properties of pharmaceutical grade AB-101. Chen uses a bioassay used to measure the incorporation rate of H-thymidine into the DNA of the cells in the presence of the test sample. This bioassay provides a measure of the wound healing property of the “sap.” Chen uses the Croton lechleri Müll. Arg latex from Ecuador. This assay indicated that the dried sap and MeOH Extract would have an incorporation rate of 68+/−12 and 88=/−5. According to Chen the dried sap and Me0H was found to be very inhibitory to wound healing properties. One familiar in the art would not assume that a Croton lechleri Müll. Arg latex extract as a whole would be effective in wound healing properties. Further, Chen states that the Ecuador sap contained only traces of taspine. Chen wanted to completely minimize or eliminate taspine due to the concern of being cytotoxin. Chen evaluated specific compound through extraction. Specifically, in the case of gallocatechin and epigalocatechin were rated as slightly stimulating to cell proliferation, while Catechin and Epicatechin showed little effect. Further Chen states that taspine and dimethylcedrusin showed little healing effects in the Ecuadorian sap.

The pharmaceutical grade of AB-101 identified a unique composition to maximize the healing properties while maintaining the film forming, low LogP and antibiotic activity. While Chen would not use the whole Croton lechleri Müll. Arg latex containing taspine or dimethylcedrusin, AB-101 pharmaceutical grade maintained using the entire Croton lechleri Müll. Arg latex in the composition for medicinal benefits associated with a topical wound healing benefits. Taspine has antibiotic, antiviral and anti-inflammatory properties. Dimethylcedrusin has unique fibroblast stimulating properties to promote healing. Taspine was targeted at least about 45 PPM and dimethylcedrusin was targeted to have a detectable presence be at least about 0.1 PPM. gallocatechin and epigallocatechin were optimized to have a combined total composition of at least about 60% of the total 4 catechins where epigallocatechin was to have a composition at least about 45% of the total 4 catechins.

The excipient(s) must be “acceptable” in the sense of being compatible with the other ingredients of the formulation and not deleterious to the recipient thereof. The excipient(s) will utilize a low number of known, well-characterized excipient ingredients that will not impart irritation or sensitization when used topically or in wounds or reduce the efficacy of AB-101. Proper formulation of the pharmaceutical composition is dependent upon the route of administration chosen. Any of the well-known techniques and excipients may be used as suitable and as understood in the art. The pharmaceutical compositions disclosed herein may be manufactured in any manner known in the art.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, or ionic liquids. The pharmaceutical compositions can additionally include: lubricating agents such as talc, magnesium stearate, and mineral oil; wetting agents; emulsifying and suspending agents; preserving agents such as methyl- and propylhydroxy-benzoates.

The compositions include those suitable for topical (including, for example, dermal, buccal, sublingual, intraocular, and wound cavity) although the most suitable route may depend upon for example the condition and disorder of the recipient. The compositions may conveniently be presented in unit dosage form and may be prepared by any of the methods well known in the art of pharmacy. Typically, these methods include the step of bringing into association latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, disclosed herein (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition.

The pharmaceutical compositions disclosed herein may be administered topically, that is by non-systemic administration. This includes the application of a compound disclosed herein externally to the surface of the skin and/or to the wound cavity and to achieve therapeutically effective amounts in the skin, such as the epidermis, dermis and/or wound cavity. In embodiments, topical administration or a topical pharmaceutical composition does not result in systemic administration or systemic exposure of the Croton lechleri to the patient.

In some embodiments, pharmaceutical compositions suitable for topical administration include liquid or semi-liquid preparations suitable for penetration through the skin to the site of inflammation such as a solution, powder, fluid emulsion, fluid suspension, semi-solid, ointment, paste, cream, gel, jelly, foam, liniment, lotion, drops, aerosols, and sprays.

Lotions include those suitable for application to the skin. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid pharmaceutical compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base.

Preferred unit dosage pharmaceutical compositions are those containing an effective dose, as herein below recited, or an appropriate fraction thereof, of the active ingredient.

When employed as pharmaceuticals, the compounds can be administered in the form of pharmaceutical compositions. These compositions can be prepared in a manner well known in the pharmaceutical arts, and can be administered by a variety of routes, depending upon whether local or systemic treatment is desired and upon the area to be treated. Administration of the disclosed compounds or compositions may be topical (including dermal, buccal, sublingual, intraocular, and wound cavity). Pharmaceutical compositions for topical administration may include foams, transdermal patches, ointments, lotions, creams, gels, solutions, fluid emulsions, fluid suspensions, semi-solids, pastes, drops, suppositories, aerosols, sprays, liquids and powders. Conventional pharmaceutical carriers, aqueous, powder or oily bases, thickeners and the like may be necessary or desirable. In some embodiments, the compounds can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance.

In certain embodiments, the pharmaceutical composition is not a soap.

In certain embodiments, the pharmaceutical composition is an ointment.

The pharmaceutical compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active pharmaceutical compositions comprising latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, can be effective over a wide dosage range and can be generally administered in a therapeutically effective amount. It will be understood, however, that the amount of the pharmaceutical compositions comprising latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the pharmaceutical composition may comprise about 0.01% to about 50% of latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, disclosed herein. In some embodiments, the latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 300%, about 1% to about 295%, about 1% to about 290%, about 1% to about 285%, about 1% to about 280%, about 1% to about 275%, about 1% to about 270%, about 1% to about 265%, about 1% to about 260%, about 1% to about 255%, about 1% to about 250%, about 1% to about 245%, about 1% to about 240%, about 1% to about 235%, about 1% to about 230%, about 1% to about 225%, about 1% to about 220%, about 1% to about 215%, about 1% to about 210%, about 1% to about 205%, about 1% to about 200%, 195%, about 1% to about 190%, about 1% to about 185%, about 1% to about 180%, about 1% to about 175%, about 1% to about 170%, about 1% to about 165%, about 1% to about 160%, about 1% to about 155%, about 1% to about 150%, about 1% to about 145%, about 1% to about 140%, about 1% to about 135%, about 1% to about 130%, about 1% to about 125%, about 1% to about 120%, about 1% to about 115%, about 1% to about 110%, about 1% to about 105%, about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 2% to about 300%, about 2% to about 295%, about 2% to about 290%, about 2% to about 285%, about 2% to about 280%, about 2% to about 275%, about 2% to about 270%, about 2% to about 265%, about 2% to about 260%, about 2% to about 255%, about 2% to about 250%, about 2% to about 245%, about 2% to about 240%, about 2% to about 235%, about 2% to about 230%, about 2% to about 225%, about 2% to about 220%, about 2% to about 215%, about 2% to about 210%, about 2% to about 205%, about 2% to about 200%, 195%, about 2% to about 190%, about 2% to about 185%, about 2% to about 180%, about 2% to about 175%, about 2% to about 170%, about 2% to about 165%, about 2% to about 160%, about 2% to about 155%, about 2% to about 150%, about 2% to about 145%, about 2% to about 140%, about 2% to about 135%, about 2% to about 130%, about 2% to about 125%, about 2% to about 120%, about 2% to about 115%, about 2% to about 110%, about 2% to about 105%, about 2% to about 100%, about 2% to about 95%, about 2% to about 90%, about 2% to about 85%, about 2% to about 80%, about 2% to about 75%, about 2% to about 70%, about 2% to about 65%, about 2% to about 60%, about 2% to about 55%, about 2% to about 50%, about 2% to about 45%, about 2% to about 40%, about 2% to about 35%, about 2% to about 30%, about 2% to about 25%, about 2% to about 20%, about 2% to about 15%, about 3% to about 300%, about 3% to about 295%, about 3% to about 290%, about 3% to about 285%, about 3% to about 280%, about 3% to about 275%, about 3% to about 270%, about 3% to about 265%, about 3% to about 260%, about 3% to about 255%, about 3% to about 250%, about 3% to about 245%, about 3% to about 240%, about 3% to about 235%, about 3% to about 230%, about 3% to about 225%, about 3% to about 220%, about 3% to about 215%, about 3% to about 210%, about 3% to about 205%, about 3% to about 200%, 195%, about 3% to about 190%, about 3% to about 185%, about 3% to about 180%, about 3% to about 175%, about 3% to about 170%, about 3% to about 165%, about 3% to about 160%, about 3% to about 155%, about 3% to about 150%, about 3% to about 145%, about 3% to about 140%, about 3% to about 135%, about 3% to about 130%, about 3% to about 125%, about 3% to about 120%, about 3% to about 115%, about 3% to about 110%, about 3% to about 105%, about 3% to about 100%, about 3% to about 95%, about 3% to about 90%, about 3% to about 85%, about 3% to about 80%, about 3% to about 75%, about 3% to about 70%, about 3% to about 65%, about 3% to about 60%, about 3% to about 55%, about 3% to about 50%, about 3% to about 45%, about 3% to about 40%, about 3% to about 35%, about 3% to about 30%, about 3% to about 25%, about 3% to about 20%, about 3% to about 15%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or a range between any two of these values. The forgoing percentages are relative to a composition made from AB-101 with exemplary amounts of the marker compounds present in the latex as disclosed in Table 1a. To illustrate, a pharmaceutical composition comprising 100% of AB-101 will contain at least about 110 PPM of gallocatechin, while a pharmaceutical composition comprising 200% of AB-101 will contain at least about 220 PPM of gallocatechin. The foregoing all representing weight percentages of embodiments of the pharmaceutical compositions. In some embodiments, the pharmaceutical composition is suitable for topical administration (including, for example, dermal, buccal, sublingual, intraocular, and wound cavity).

In some embodiments, the latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, is in a therapeutically effective amount. In some embodiments, the therapeutically effective amount may be in an amount of about 0.01% to about 100%, about 0.01% to about 95%, about 0.01% to about 90%, about 0.01% to about 85%, about 0.01% to about 80%, about 0.01% to about 75%, about 0.01% to about 70%, about 0.01% to about 65%, about 0.01% to about 60%, about 0.01% to about 55%, about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 100%, about 0.05% to about 95%, about 0.05% to about 90%, about 0.05% to about 85%, about 0.05% to about 80%, about 0.05% to about 75%, about 0.05% to about 70%, about 0.05% to about 65%, about 0.05% to about 60%, about 0.05% to about 55%, about 0.05% to about 50%, about 0.05% to about 45%, about 0.05% to about 40%, about 0.05% to about 30%, about 0.05% to about 20%, about 0.05% to about 10%, about 0.1% to about 100%, about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 300%, about 1% to about 295%, about 1% to about 290%, about 1% to about 285%, about 1% to about 280%, about 1% to about 275%, about 1% to about 270%, about 1% to about 265%, about 1% to about 260%, about 1% to about 255%, about 1% to about 250%, about 1% to about 245%, about 1% to about 240%, about 1% to about 235%, about 1% to about 230%, about 1% to about 225%, about 1% to about 220%, about 1% to about 215%, about 1% to about 210%, about 1% to about 205%, about 1% to about 200%, 195%, about 1% to about 190%, about 1% to about 185%, about 1% to about 180%, about 1% to about 175%, about 1% to about 170%, about 1% to about 165%, about 1% to about 160%, about 1% to about 155%, about 1% to about 150%, about 1% to about 145%, about 1% to about 140%, about 1% to about 135%, about 1% to about 130%, about 1% to about 125%, about 1% to about 120%, about 1% to about 115%, about 1% to about 110%, about 1% to about 105%, about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 2% to about 300%, about 2% to about 295%, about 2% to about 290%, about 2% to about 285%, about 2% to about 280%, about 2% to about 275%, about 2% to about 270%, about 2% to about 265%, about 2% to about 260%, about 2% to about 255%, about 2% to about 250%, about 2% to about 245%, about 2% to about 240%, about 2% to about 235%, about 2% to about 230%, about 2% to about 225%, about 2% to about 220%, about 2% to about 215%, about 2% to about 210%, about 2% to about 205%, about 2% to about 200%, 195%, about 2% to about 190%, about 2% to about 185%, about 2% to about 180%, about 2% to about 175%, about 2% to about 170%, about 2% to about 165%, about 2% to about 160%, about 2% to about 155%, about 2% to about 150%, about 2% to about 145%, about 2% to about 140%, about 2% to about 135%, about 2% to about 130%, about 2% to about 125%, about 2% to about 120%, about 2% to about 115%, about 2% to about 110%, about 2% to about 105%, about 2% to about 100%, about 2% to about 95%, about 2% to about 90%, about 2% to about 85%, about 2% to about 80%, about 2% to about 75%, about 2% to about 70%, about 2% to about 65%, about 2% to about 60%, about 2% to about 55%, about 2% to about 50%, about 2% to about 45%, about 2% to about 40%, about 2% to about 35%, about 2% to about 30%, about 2% to about 25%, about 2% to about 20%, about 2% to about 15%, about 3% to about 300%, about 3% to about 295%, about 3% to about 290%, about 3% to about 285%, about 3% to about 280%, about 3% to about 275%, about 3% to about 270%, about 3% to about 265%, about 3% to about 260%, about 3% to about 255%, about 3% to about 250%, about 3% to about 245%, about 3% to about 240%, about 3% to about 235%, about 3% to about 230%, about 3% to about 225%, about 3% to about 220%, about 3% to about 215%, about 3% to about 210%, about 3% to about 205%, about 3% to about 200%, 195%, about 3% to about 190%, about 3% to about 185%, about 3% to about 180%, about 3% to about 175%, about 3% to about 170%, about 3% to about 165%, about 3% to about 160%, about 3% to about 155%, about 3% to about 150%, about 3% to about 145%, about 3% to about 140%, about 3% to about 135%, about 3% to about 130%, about 3% to about 125%, about 3% to about 120%, about 3% to about 115%, about 3% to about 110%, about 3% to about 105%, about 3% to about 100%, about 3% to about 95%, about 3% to about 90%, about 3% to about 85%, about 3% to about 80%, about 3% to about 75%, about 3% to about 70%, about 3% to about 65%, about 3% to about 60%, about 3% to about 55%, about 3% to about 50%, about 3% to about 45%, about 3% to about 40%, about 3% to about 35%, about 3% to about 30%, about 3% to about 25%, about 3% to about 20%, about 3% to about 15%, about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 3%, about 5%, about 10%, about 15%, about 20%, about 25%, about 30%, about 35%, about 40%, about 45%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or a range between any two of these values. The forgoing percentages are relative to a composition made from AB-101 with exemplary amounts of the marker compounds present in the latex as disclosed in Table 1a. To illustrate, a therapeutically effective amount in the amount of 100% of AB-101 will contain at least about 110 PPM of gallocatechin, while a therapeutically effective amount in the amount of 200% of AB-101 will contain at least about 220 PPM of gallocatechin. The foregoing all representing weight percentages of the pharmaceutical composition.

In some embodiments, the therapeutically effective amount can vary according to, for example, the particular use for which the treatment is made, the manner of administration of the compound, the health and condition of the patient, and the judgment of the prescribing physician. The proportion or concentration of latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, in a pharmaceutical composition can vary depending upon a number of factors including dosage, chemical characteristics (e.g., hydrophobicity), and the route of administration. For example, the compounds can be provided in an aqueous physiological buffer solution containing about 0.1 to about 10% w/v of the compound for parenteral administration. Some typical dose ranges for the compounds are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, composition of the excipient, and its route of administration. Effective doses can be extrapolated from dose-response curves derived from in vitro or animal model test systems.

The amount of composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

In some embodiments, the present invention of any of the embodiments disclosed herein may comprise one or more additional blood clotting agents. Specific, non-limiting examples of possible additional blood clotting agents include, but not limited to, hemostatic agents and super absorbing polymers.

Specific, non-limiting examples of hemostatic agents include, but not limited to, factor concentrators, mucoadhesive agents, and procoagulant supplementors.

Super absorbing polymers may be made by employing methods known in the art. Specific, non-limiting examples of super absorbing polymers include, but not limited to, sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

In some embodiments, the pharmaceutical compositions of the present invention is first applied to a bandage (e.g., gauze, wound dressing) to form a treated bandage, which is then applied to wounds in a subject. In some embodiments, the pharmaceutical compositions of the present invention is first impregnated into a bandage (e.g., gauze, wound dressing) to form a treated bandage, which is then applied to wounds in a subject.

In some embodiments, a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard is first applied to a bandage (e.g., gauze, wound dressing) to form a treated bandage, which is then applied to wounds in a subject. In some embodiments, a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard is first impregnated into a bandage (e.g., gauze, wound dressing) to form a treated bandage, which is then applied to wounds in a subject.

In some embodiments, the additional blood clotting agent of any of the embodiments herein may be applied to a bandage (e.g., gauze, wound dressing) before, after, or concomitantly with any of the pharmaceutical compositions of the present invention disclosed herein to form a treated bandage, which is then applied to wounds in a subject. In some embodiments, the additional blood clotting agent of any of the embodiments herein may be impregnated into a bandage (e.g., gauze, wound dressing) before, after, or concomitantly with any of the pharmaceutical compositions of the present invention disclosed herein to form a treated bandage, which is then applied to wounds in a subject.

In some embodiments, the additional blood clotting agent of any of the embodiments herein may be applied to a bandage (e.g., gauze, wound dressing) before, after, or concomitantly with a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard to form a treated bandage, which is then applied to wounds in a subject. In some embodiments, the additional blood clotting agent of any of the embodiments herein may be impregnated into a bandage (e.g., gauze, wound dressing) before, after, or concomitantly with a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard to form a treated bandage, which is then applied to wounds in a subject.

### Methods of Use

The present invention relates to a method of treatment wounds in a subject, treating or preventing or reducing the risk of a bacterial infection of wounds in a subject, and/or inducing blood clotting of a wound in a subject comprising the administration of a treated bandage comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, or a treated bandage comprising a pharmaceutical composition containing latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein. In embodiments, the pharmaceutical composition may include a pharmaceutically acceptable excipient.

Also provided herein is a treated bandage comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein for use as a medicament.

Also provided herein is a treated bandage comprising a pharmaceutical composition containing latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein for use as a medicament.

Also provided herein is a treated bandage comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein for use as a medicament for treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

Also provided herein is a treated bandage comprising a pharmaceutical composition containing latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein for use as a medicament for the treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

Also provided is a treated bandage comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein, for use in the manufacture of a medicament for the treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

Also provided is a treated bandage comprising a pharmaceutical composition containing latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein for use in the manufacture of a medicament for the treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

The treated bandage of any of the embodiments disclosed herein may promote wound healing in a subject.

The treated bandage of any of the embodiments disclosed herein may promote wound occlusion in a subject.

The treated bandage of any of the embodiments disclosed herein may promote wound occlusion thereby reducing scarring in a subject.

The treated bandage of any of the embodiments disclosed herein may promote tissue regeneration in a subject.

The treated bandage of any of the embodiments disclosed herein may promote tissue growth in a subject.

The treated bandage of any of the embodiments disclosed herein may promote tissue growth thereby accelerating wound healing in a subject.

The treated bandage of any of the embodiments disclosed herein may promote blood clotting, blood coagulation, clot formation, or any combination thereof, in a subject.

The treated bandage of any of the embodiments disclosed herein may promote blood clotting by accelerating the normal platelet clotting pathway in a subject.

Also provided is the use of a treated bandage comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein.

Also provided is use of a treated bandage comprising a pharmaceutical composition containing latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein.

In some embodiments, the use is selected from the group consisting of treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

In some embodiments, the use is treatment of wounds in a subject.

In some embodiments, the use is treatment of chronic wounds in a subject.

In some embodiments, the use is treatment of traumatic wounds in a subject.

In some embodiments, the use is treatment of acute wounds in a subject,

In some embodiments, the use is treating or preventing or reducing the risk of a bacterial infection of wounds in a subject.

In some embodiments, the use is inducing blood clotting of a wound in a subject.

In some embodiments, the use is promotion of blood coagulation in a subject.

In some embodiments, the use is promotion of clot formation in a subject.

In some embodiments, the use is promotion of blood clotting by accelerating the normal platelet clotting pathway in a subject.

In some embodiments, the use is promotion of wound healing in a subject.

In some embodiments, the use is promotion of wound occlusion in a subject.

In some embodiments, the use is promotion of wound occlusion thereby reducing scarring in a subject.

In some embodiments, the use is promotion of tissue regeneration in a subject.

In some embodiments, the use is promotion of tissue growth in a subject.

In some embodiments, the use is promotion of tissue growth thereby accelerating wound healing in a subject.

Also provided herein is a method of treating a condition in a subject comprising contacting the subject in need thereof with a treated bandage comprising a therapeutically effective amount of latex of Croton lechleri, preferably filtered latex of Croton lechleri, preferably filtered latex of Croton lechleri Müll.Arg., wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard.

Also provided herein is a method of treating a condition in a subject comprising contacting the subject in need thereof with a treated bandage comprising a pharmaceutical composition containing latex of Croton lechleri, preferably filtered latex of Croton lechleri, preferably filtered latex of Croton lechleri Müll.Arg., wherein the composition contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein.

In some embodiments, the condition is selected from the group consisting of treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

In some embodiments, the condition is treatment of wounds in a subject.

In some embodiments, the condition is treatment of chronic wounds in a subject.

In some embodiments, the condition is treatment of traumatic wounds in a subject.

In some embodiments, the condition is treatment of acute wounds in a subject,

In some embodiments, the condition is treating or preventing or reducing the risk of a bacterial infection of wounds in a subject.

In some embodiments, the condition is inducing blood clotting of a wound in a subject.

In some embodiments, the condition is promotion of blood coagulation in a subject.

In some embodiments, the condition is promotion of clot formation in a subject.

In some embodiments, the condition is promotion of blood clotting by accelerating the normal platelet clotting pathway in a subject.

In some embodiments, the condition is promotion of wound healing in a subject.

In some embodiments, the condition is promotion of wound occlusion in a subject.

In some embodiments, the condition is promotion of wound occlusion thereby reducing scarring in a subject.

In some embodiments, the condition is promotion of tissue regeneration in a subject.

In some embodiments, the condition is promotion of tissue growth in a subject.

In some embodiments, the condition is promotion of tissue growth thereby accelerating wound healing in a subject.

In any embodiments disclosed herein, chronic wounds includes, but not limited to, venous ulcer wounds, arterial ulcer wounds, mixed venous and arterial ulcer wounds, decubitus ulcer wounds (a.k.a. bed sores, a.k.a. pressure ulcers), diabetic ulcers, or a combination thereof.

The treated bandage is applied to each wound. If the bandage is separated from the wound or if the bandage has been worn for 24 hours, a new, treated bandage should be applied. A new bandage is generally applied every day. In one embodiment, the composition is administered until the symptoms (e.g., wounds) disappear, become less pronounced, or problematic side effects occur.

The treated bandage of any of the embodiments disclosed herein may applied externally to the surface of the skin, to the wound cavity, inside the wound cavity, or any combination thereof.

The pharmaceutical compositions may be administered in various modes, e.g. topical (including, for example, dermal, buccal, sublingual, intraocular, and wound cavity). The specific dose level for any particular patient will depend upon a variety of factors including the activity of the specific compound employed, the age, body weight, general health, sex, diet, time of administration, route of administration, rate of excretion, drug combination, the precise disorder being treated, and the severity of the indication or condition being treated. Also, the route of administration may vary depending on the condition and its severity.

Pharmaceutical compositions of the present invention may be administered once per day, twice per day, thrice per day, 4 times per day, 5 times per day, 6 times per day, 7 times per day, 8 times per day, 9 times per day, 10 times per day, or a range between of these values. In particular embodiments, the pharmaceutical compositions is administered twice per day.

Pharmaceutical compositions of the present invention may be administered continuously, every 15 minutes 30 min., 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In particular embodiments, the administration lasts 24 weeks. In particular embodiments, the administration lasts 2 weeks.

Treatment of the target wound may last 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values.

Treatment of the wound may continue until complete resolution of the target wound.

Treatment of the wounds in a subject, treating or preventing or reducing the risk of a bacterial infection of wounds in a subject, and/or inducing blood clotting of a wound in a subject may continue at the discretion of the prescribing physician.

In certain embodiments, the pharmaceutical compositions of the present invention may be topically applied directly to a wound in a subject in order to treat them, treat or prevent infection of the wound in a subject, and/or induce blood clotting of the wound in a subject.

In some embodiments, the pharmaceutical composition has a minimum inhibitory concentration (MIC) of at least about 0.01%, at least about 0.05%, at least about 0.1%, at least about 0.25%, at least about 0.5%, at least about 0.75%, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%.

### Example 1—LogP Studies with AB-101

LogP measures the lipophilicity of a molecule indicating how readily an analyte can partition between aqueous and organic phases and thus can be used as a predictor to understand drug behavior in the body. It is a determining factor for a compound's absorption, distribution, and penetration across biological membranes. A more polar, hydrophilic compound will have a lower LogP and thus preference for the aqueous phase. Non-polar, hydrophobic compounds will have higher LogP values and prefer partitioning into the organic phase. Based on the AB-101 LogP values, Alphyn will determine the distribution of the 4 PACs and taspine into skin tissue and systemic circulation. In a topically delivered product, a low LogP leads to increased susceptibility to aqueous solubility, lower lipophilicity, and therefore poor distribution in the body. As a result, we would expect membrane permeability to be poor. This would generally support the conclusions in section in that AB-101 dosing would not exhibit high permeation thus keeping systemic levels low or insignificant.

The LogP assessment revealed the compounds of interest to possess more polar properties, good aqueous solubility, poor lipid solubility, and poor adsorption and distribution properties as defined by LogP (Table 3). These values were all below 2, predicting poor disruption of these compounds in the resin.

TABLE 3
LogP Values

| | Log P | Log P | Log P | Log P | Log P |
|---|---|---|---|---|---|
| | Catechin | Epicatechin | Gallocatechin | Epigallocatechin | Taspine |
| | (0.51-1.8)* | (0.4-1.8)* | (0.71-1.41)* | (0.00-1.49)* | (0.99-1.8)* |
| Lot 0 | 0.27 | −0.09 | −0.24 | −0.79 | −1.15 |
| Lot 0 - resuspended | 0.39 | 0.02 | 0.36 | 1.20 | −1.06 |
| Lot 1 | 0.29 | −0.02 | 0.01 | 0.27 | −1.76 |
| Lot 1 - resuspended | 0.23 | 0.02 | −0.13 | −0.11 | −0.94 |
| Lot 2 | 0.59 | 0.13 | 0.56 | 0.86 | −0.90 |
| Lot 2 - resuspended | 0.07 | −0.17 | −0.48 | −1.71 | −1.71 |
| Lot X | −0.14 | 0.07 | 0.03 | 1.82 | −0.31 |
| Lot X - resuspended | −0.13 | 0.09 | −0.25 | −0.55 | −0.07 |

*Reference standard LogP values from ALOGPS, ChemAxon, and Chemspider
Resuspended means lyophilized AB-101 was resuspended in HPLC-grade methanol

### Example 2—Skin Permeation Studies with AB-101

These experiments were designed to assess the in vitro partitioning and transdermal release of catechin, epicatechin, gallocatechin, epigallocatechin, and taspine from AB-101 100% based on the in vitro release testing and partitioning assays. In vitro testing such as this is an accepted, industry-standard method for determining the skin penetration of a topically applied products (USP Chapter 1724, USP 36/NF31, 2013), and in vitro testing using Strat-M membranes has been shown to be predictive of in vivo results (Flaten et al, 2005). The Franz diffusion cell model is a well establish industry and academic method for performing permeation testing (Ueda et al, 2009; Li & Rahn, 1993). To determine the flux of catechin, epicatechin, gallocatechin, epigallocatechin, and taspine into and through the skin, an existing analytical LC-MS/MS method was used.

In vitro release testing (IVRT) data revealed poor transdermal release and permeation of these compounds from AB-101 100%. As defined by flux, AB-101 lots (X and 1) demonstrated low amounts permeating across all 5 compounds tested. Results are shown in Table 4.

TABLE 4
Flux Calculations

| Vehicle | Catechin | Epicatechin | Gallocatechin | Epigallocatechin | Taspine |
|---|---|---|---|---|---|
| Lot 1 | 8.82 μg/cm²/hr | 9.11 μg/cm²/hr | 18.25 μg/cm²/hr | 503.51 μg/cm²/hr | 10.01 μg/cm²/hr |
| Lot X | 1.53 μg/cm²/hr | 1.04 μg/cm²/hr | 4.33 μg/cm²/hr | 57.09 μg/cm²/hr | 16.69 μg/cm²/hr |

### Summary of the Results of Examples 1 and 2

Taken together, the results indicate that the 4 proanthocyanidins and taspine demonstrate low accumulation, flux and permeability across intact synthetic membranes. Any permeation that does occur would be unlikely to distribute at significant levels due to the poor distribution properties revealed from the in vitro LogP analysis. The results of these studies support the claim that the 4 proanthocyanidins and taspine measured are unlikely to reach toxic amounts at a systemic level based on these in vitro results.

When AB-101 is applied to an open wound the goal is to have very little absorption or partitioning into the wound and blood stream. This provides the Pharmaceutical grade of AB-101 with a strong safety profile. The data support the conclusion that the likelihood of topical AB-101 permeating the skin layers in any appreciable amount is extremely low. The data also indicate that any AB-101 that penetrates the skin will likely be metabolized within the skin prior to having an opportunity for systemic absorption. Furthermore, the likelihood of the AB-101 constituents being absorbed systemically were they to penetrate is also low given the logP values, which predict little or no passage. Shown for the first time is a pharmaceutical grade AB-101 that has both low permeability to enter into the blood stream and be absorbed by the tissue and organs leading to a safety concern.

### Example 3—Antibacterial Activity

To demonstrate the effectiveness of AB-101 and how changes within different type of latex extract can change performance and well as showing that not all extracts, even in the same plant species, yields the same pharmaceutical grade performance, invitro assay of MSSA and MRSA were conducted. Measures included the minimum inhibitory concentration (MIC) which is the lowest concentration of AB-101 that prevents visible growth of the bacterium or pathogen, and minimum bactericidal concentration (MBC) which is the lowest concentration of an antibacterial agent required to kill a bacterium.

The comparison of AB-101 Lot 01 and 2 for MIC demonstrates a high effectiveness against MSSA and MRSA with particular emphasis on the Mupirocin resistant strain of MRSA. Mupirocin is a leading topical treatment for MRSA. Shown for the first time is the effectiveness of AB-101 against these pathogens and importantly an improvement over the leading current pharmaceutical treatment. Results are shown in Table 5.

TABLE 5

| | | MIC (% vol./vol.) | | | | MIC (μg/mL) | |
|---|---|---|---|---|---|---|---|
| Strain ID | Characteristic | AB-101 Lot 01 | | AB-101 Lot 02 | | Methicillin | Mupirocin |
| CDC 218 | MupirocinR, MRSA | 12.5 | 12.5 | 12.5 | 12.5 | >64 | >256 |
| CDC 224 | MupirocinR, MRSA | 12.5 | 12.5 | 12.5 | 12.5 | >64 | >256 |
| CDC 228 | MupirocinR, MRSA | 3.13 | 3.13 | 3.13 | 3.13 | 64 | >256 |
| 1674606 | MupirocinR, MSSA | 50 | 50 | 50 | 50 | 2-8 | >256 |
| 1674607 | MupirocinR, MRSA | 12.5 | 12.5 | 12.5 | 12.5 | >64 | >256 |
| 1674608 | MupirocinR, MSSA | 12.5 | 12.5 | 25 | 50 | 2-4 | >256 |
| 1674611 | MupirocinR, MRSA | 6.25 | 6.25 | 12.5 | 12.5 | 64 | >256 |
| CDC 480 | MupirocinS, MRSA | 12.5 | 25 | 12.5 | 25 | 32-64 | ≤0.25 |
| CDC 481 | MupirocinS, MRSA | 12.5 | 12.5 | 12.5 | 12.5 | 16-32 | ≤0.25 |
| CDC 482 | MupirocinS, MRSA | 12.5 | 12.5 | 12.5 | 12.5 | 8-32 | ≤0.25 |
| CDC 483 | MupirocinS, MRSA | 12.5 | 12.5 | 12.5 | 12.5 | 64 | ≤0.25 |
| CDC 220 | MupirocinS, MRSA | 12.5 | 12.5 | 6.25 | 12.5 | 64->64 | ≤0.25 |
| CDC 227 | MupirocinS, MRSA | 6.25 | 6.25 | 12.5 | 12.5 | 64 | ≤0.25-0.5 |
| CDC 461 | MupirocinS, MSSA | 12.5 | 12.5 | 12.5 | 12.5 | 4-8 | ≤0.25-1 |
| CDC 462 | MupirocinS, MSSA | 6.25 | 12.5 | 6.25 | 12.5 | 8 | ≤0.25 |
| CDC 463 | MupirocinS, MSSA | 6.25 | 6.25 | 12.5 | 12.5 | 8-16 | ≤0.25-0.5 |
| CDC 464 | MupirocinS, MSSA | 12.5 | 12.5 | 12.5 | 12.5 | 2 | ≤0.25 |
| CDC 484 | MupirocinS, MSSA | 12.5 | 12.5 | 12.5 | 12.5 | 2 | ≤0.25 |
| CDC 485 | MupirocinS, MSSA | 50 | 50 | 50 | 50 | 2-4 | ≤0.25 |
| ATCC 29213 | QC control | 12.5 | 50-12.5 | 12.5 | 12.5 | 1-2 | ≤0.25-0.5 |

Table 6 shows the comparison of AB-101 Lots 01 and 02 for MBC demonstrates a high effectiveness against MSSA and MRSA with particular emphasis on the mupirocin resistant strain of MRSA. Once again Mupirocin is a leading topical treatment for MRSA. Shown for the first time is the effectiveness of AB-101 against these pathogens and importantly an improvement over the leading current pharmaceutical treatment.

TABLE 6

| | | MBC (% vol./vol.) | | | |
|---|---|---|---|---|---|
| Strain | | AB-101 | | AB-101 | |
| ID | Characteristic | Lot 1 | | Lot 2 | |
| CDC 218 | MupirocinR, MRSA | 50 | 50 | 50 | 50 |
| CDC 224 | MupirocinR, MRSA | 50 | 50 | 50 | 50 |
| CDC 228 | MupirocinR, MRSA | 6.25 | 6.25 | 6.25 | 6.25 |
| 1674606 | MupirocinR, MSSA | >50 | >50 | >50 | >50 |
| 1674607 | MupirocinR, MRSA | 12.5 | 12.5 | 25 | 25 |
| 1674608 | MupirocinR, MSSA | 50 | 50 | 50 | >50 |
| 1674611 | MupirocinR, MRSA | 12.5 | 12.5 | 50 | 50 |
| CDC 480 | MupirocinS, MRSA | 50 | 50 | 50 | 50 |
| CDC 481 | MupirocinS, MRSA | 50 | 50 | 50 | 50 |
| CDC 482 | MupirocinS, MRSA | 50 | 50 | 50 | 50 |
| CDC 483 | MupirocinS, MRSA | 50 | 50 | 50 | 50 |
| CDC 220 | MupirocinS, MRSA | 25 | 25 | 25 | 25 |
| CDC 227 | MupirocinS, MRSA | 12.5 | 12.5 | 25 | 25 |
| CDC 461 | MupirocinS, MSSA | 50 | 50 | 50 | 50 |
| CDC 462 | MupirocinS, MSSA | 12.5 | 50 | 50 | 50 |
| CDC 463 | MupirocinS, MSSA | 12.5 | 12.5 | 12.5 | 12.5 |
| CDC 464 | MupirocinS, MSSA | >50 | 50 | 50 | 50 |
| CDC 484 | MupirocinS, MSSA | 50 | 50 | 50 | 50 |
| CDC 485 | MupirocinS, MSSA | 50 | 50 | 50 | 50 |
| ATCC | QC control | 50-12.5 | 50-12.5 | 50 | 50 |
| 29213 | | | | | |

Table 7 compares AB-101 lot X and Lot 00 for MIC because these lots have been shown elsewhere to have different composition. Lot X and Lot 00 are latex extracts of Croton lechleri Müll.Arg., the same species, grown in similar locations. Lot X demonstrates a significantly higher effectiveness against MSSA and MRSA. This data shows for the first time that not all latex extracts of Croton lechleri Müll.Arg. provide the same performance, even when the extracts are from the same species grown in similar locations.

TABLE 7

| | | MIC (% vol./vol.) | | | | MIC (μg/mL) | |
|---|---|---|---|---|---|---|---|
| Strain ID | Characteristic | AB-101 Lot 00 | | AB-101 Lot X | | Methicillin | Mupirocin |
| CDC 218 | MupirocinR, MRSA | 12.5 | 12.5 | 0.78 | 0.78 | >64 | >256 |
| CDC 224 | MupirocinR, MRSA | 12.5 | 12.5 | 0.78 | 0.78 | >64 | >256 |
| CDC 228 | MupirocinR, MRSA | 3.13 | 3.13 | 0.39 | 0.39 | 64 | >256 |
| 1674606 | MupirocinR, MSSA | >50 | >50 | 1.56 | 1.56 | 2-8 | >256 |
| 1674607 | MupirocinR, MRSA | 12.5 | 12.5 | 0.78 | 0.78 | >64 | >256 |
| 1674608 | MupirocinR, MSSA | 25 | 25 | 1.56 | 1.56 | 2-4 | >256 |
| 1674611 | MupirocinR, MRSA | 25 | 25 | 0.78 | 0.78 | 64 | >256 |
| CDC 480 | MupirocinS, MRSA | 12.5 | 12.5 | 1.56 | 1.56 | 32-64 | ≤0.25 |
| CDC 481 | MupirocinS, MRSA | 12.5 | 12.5 | 1.56 | 1.56 | 16-32 | ≤0.25 |
| CDC 482 | MupirocinS, MRSA | 12.5 | 12.5 | 0.78 | 0.78 | 8-32 | ≤0.25 |
| CDC 483 | MupirocinS, MRSA | 12.5 | 12.5 | 0.78 | 0.78 | 64 | ≤0.25 |
| CDC 220 | MupirocinS, MRSA | 12.5 | 12.5 | 0.78 | 0.78 | 64->64 | ≤0.25 |
| CDC 227 | MupirocinS, MRSA | 12.5 | 12.5 | 0.78 | 0.78 | 64 | ≤0.25-0.5 |
| CDC 461 | MupirocinS, MSSA | 50 | 50 | 0.78 | 0.78 | 4-8 | ≤0.25-1 |
| CDC 462 | MupirocinS, MSSA | 12.5 | 12.5 | 0.78 | 0.78 | 8 | ≤0.25 |
| CDC 463 | MupirocinS, MSSA | 12.5 | 12.5 | 1.56 | 0.78 | 8-16 | ≤0.25-0.5 |
| CDC 464 | MupirocinS, MSSA | >50 | >50 | 1.56 | 1.56 | 2 | ≤0.25 |
| CDC 484 | MupirocinS, MSSA | >50 | 25 | 0.78 | 1.56 | 2 | ≤0.25 |
| CDC 485 | MupirocinS, MSSA | >50 | >50 | 0.78 | 0.78 | 2-4 | ≤0.25 |
| ATCC 29213 | QC control | 12.5 | 12.5 | 1.56 | 1.56 | 1-2 | ≤0.25-0.5 |

Table 8 compares AB-101 lot X and Lot 00 for MBC because these lots have been shown elsewhere to have different composition. Lot X and Lot 00 are latex extracts of Croton lechleri Müll.Arg., the same species, grown in similar locations. Lot X demonstrates a significantly higher effectiveness against MSSA and MRSA. This data shows for the first time that not all latex extracts of Croton lechleri Müll.Arg. provide the same performance, even when the extracts are from the same species grown in similar locations.

TABLE 8

| | | MBC (% vol./vol.) | | | |
|---|---|---|---|---|---|
| Strain | | AB-101 | | AB-101 | |
| ID | Characteristic | Lot 00 | | Lot X | |
| CDC 218 | MupirocinR, MRSA | >50 | 50 | 0.78 | 0.78 |
| CDC 224 | MupirocinR, MRSA | >50 | >50 | 0.78 | 0.78 |
| CDC 228 | MupirocinR, MRSA | 12.5 | 12.5 | 0.39 | 0.78 |
| 1674606 | MupirocinR, MSSA | >50 | >50 | 1.56 | 1.56 |
| 1674607 | MupirocinR, MRSA | 25 | 25 | >50 | >50 |
| 1674608 | MupirocinR, MSSA | >50 | >50 | 1.56 | 1.56 |
| 1674611 | MupirocinR, MRSA | 50 | >50 | 0.78 | 0.78 |
| CDC 480 | MupirocinS, MRSA | >50 | >50 | 1.56 | 3.12 |
| CDC 481 | MupirocinS, MRSA | >50 | >50 | 1.56 | 1.56 |
| CDC 482 | MupirocinS, MRSA | >50 | >50 | 0.78 | 0.78 |
| CDC 483 | MupirocinS, MRSA | >50 | >50 | 0.78 | 0.78 |
| CDC 220 | MupirocinS, MRSA | 50 | 50 | 0.78 | 1.56 |
| CDC 227 | MupirocinS, MRSA | 25 | 25 | 1.56 | 0.78 |
| CDC 461 | MupirocinS, MSSA | >50 | >50 | 0.78 | 0.78 |
| CDC 462 | MupirocinS, MSSA | 50 | 50 | 0.78 | 0.78 |
| CDC 463 | MupirocinS, MSSA | 12.5 | 12.5 | 6.25 | 6.25 |
| CDC 464 | MupirocinS, MSSA | >50 | >50 | 1.56 | 1.56 |
| CDC 484 | MupirocinS, MSSA | >50 | >50 | 1.56 | 1.56 |
| CDC 485 | MupirocinS, MSSA | >50 | >50 | >50 | >50 |
| ATCC | QC control | ≥50 | ≥50 | 6.25 | 3.13 |
| 29213 | | | | | |

To demonstrate the effectiveness of AB-101 across other pathogens and in particular across gram negative (−) pathogens, Lot 01 and 02 were tested against Pseudomonas aeruginosa. All 20 of the Pseudomonas aeruginosa tested are resistant to multiple antibiotics, thereby they all fit the definition of being Multi-Drug Resistant (MDR). Of the 20, 5 are known to be Verona integron-encoded metallo-beta-lactamase (VIM)—producing Pseudomonas aeruginosa which are drug resitant strains of Pseudomonas aeruginosa, another 5 are known to be Klebsiella pneumoniae carbapenemase (KPC) producing Pseudomonas aeruginosa strains which are drug resitant strains of Pseudomonas aeruginosa, and 4 are known to be IMP-Type Metallo-β-Lactamase (IMP))—producing Pseudomonas aeruginosa which are drug resitant strains of Pseudomonas aeruginosa. The 6 remaining strains are known to be antibiotic resistant and are listed simply as MDR strains. Measures included the minimum inhibitory concentration (MIC) which is the lowest concentration of AB-101 that prevents visible growth of the bacterium or pathogen, and minimum bactericidal concentration (MBC) which is the lowest concentration of an antibacterial agent required to kill a bacterium.

Table 9 shows the comparison of AB-101 Lot 01 and 02 for MIC demonstrates a high effectiveness against Pseudomonas aeruginosa. Shown for the first time is the effectiveness of AB-101 against these pathogens and importantly an improvement over the leading current pharmaceutical treatment.

TABLE 9

| Pseudomonas | Pseudomonas | | | | | | | | |
|---|---|---|---|---|---|---|---|---|---|
| aeruginosa | aeruginosa | MIC (% AB-101)^{a} | | | | MIC (μg/mL)^{b} | | | |
| Strain ID | Characteristic | Lot 1 | | Lot 2 | | Imipenem | | Ciprofloxacin | |
| CDC 0230 | VIM | 25 | 25 | 25 | 25 | >64 | >64 | >64 | >64 |
| CDC 0239 | VIM | 12.5 | 12.5 | 12.5 | 12.5 | >64 | >64 | 32 | 32 |
| CDC 0254 | VIM | 25 | 25 | 25 | 25 | >64 | >64 | 32 | 32 |
| CDC 0255 | VIM | 25 | 25 | 25 | 25 | >64 | >64 | 32 | 32 |
| CDC 0509 | VIM | 25 | 25 | 25 | 25 | >64 | >64 | 32 | 32 |
| CDC 0231 | KPC | 25 | 25 | 25 | 25 | >64 | >64 | >64 | >64 |
| CDC 0356 | KPC | 25 | 25 | 25 | 25 | >64 | >64 | 0.125 | 0.125 |
| CDC 0441 | KPC | 25 | 25 | 25 | 25 | >64 | >64 | 2 | 2 |
| CDC 0516 | KPC | 25 | 25 | 25 | 25 | >64 | >64 | 0.125 | 0.125 |
| CDC 0518 | KPC | 12.5 | 12.5 | 12.5 | 12.5 | >64 | >64 | >64 | >64 |
| CDC 0092 | IMP | 12.5 | 12.5 | 12.5 | 50 | >64 | >64 | 32 | 32 |
| CDC 0103 | IMP | 12.5 | 12.5 | 12.5 | 12.5 | >64 | >64 | >32 | >32 |
| CDC 0439 | IMP | 12.5 | 12.5 | 12.5 | 12.5 | >64 | >64 | 32 | 32 |
| CDC 0241 | IMP | 12.5 | 12.5 | 12.5 | 12.5 | >64 | >64 | 16 | 16 |
| CDC 0508 | MDR | 12.5 | 12.5 | 12.5 | 12.5 | 16 | 16 | 2 | 2 |
| CDC 0353 | MDR | 25 | 25 | 12.5 | 12.5 | 16 | 16 | 16 | 16 |
| CDC 0357 | MDR | 25 | 25 | 25 | 25 | 16 | 16 | 32 | 32 |
| CDC 0246 | MDR | 12.5 | 12.5 | 12.5 | 12.5 | >64 | >64 | 32 | 32 |
| CDC 0250 | MDR | 25 | 12.5 | 12.5 | 25 | >64 | >64 | 32 | 32 |
| CDC 0064 | MDR | 25 | 25 | 25 | 25 | >64 | >64 | 16 | 16 |
| ATCC 27853 | QC strain | 25 | 25 | 25 | 25 | 2 | 2 | 0.25 | 0.25 |

Table 10 shows the comparison of Iminipenem and Cripofloxacin for MIC against quality control strain ATCC 27853.

TABLE 10

| | |
|---|---|
| Pseudomonas | |
| aeruginosa | CLSI QC Range MIC (μg/ml) |

| Strain | Imipenem | Ciprofloxacin |
|---|---|---|
| ATCC 27853 | 1-4 | 0.125-1 |

Table 11 shows the comparison of AB-101 Lots 01 and 02 for MBC demonstrates a high effectiveness against Pseudomonas aeruginosa. Shown for the first time is the effectiveness of AB-101 against these pathogens specifically for the multi-drug resistant pathogens.

TABLE 11

| Pseudomonas | | | | | | |
|---|---|---|---|---|---|---|
| aeruginosa | Pseudomonas | Pseudomonas | | | | |
| screen | aeruginosa | aeruginosa | MBC (% AB-101) | | | |
| number | ID | Characteristic | Lot 01 | | Lot 02 | |
| 1 | CDC 0230 | VIM | 25 | 25 | 25 | 25 |
| 2 | CDC 0239 | VIM | 25 | 25 | 25 | 25 |
| 3 | CDC 0254 | VIM | 25 | 25 | 25 | 25 |
| 4 | CDC 0255 | VIM | 25 | 25 | 25 | 25 |
| 5 | CDC 0509 | VIM | 25 | 25 | 25 | 25 |
| 6 | CDC 0231 | KPC | 25 | 25 | 25 | 25 |
| 7 | CDC 0356 | KPC | 25 | 25 | 25 | 25 |
| 8 | CDC 0441 | KPC | 25 | 25 | 25 | 25 |
| 9 | CDC 0516 | KPC | 25 | 25 | 25 | 25 |
| 10 | CDC 0518 | KPC | 25 | 25 | 25 | 25 |
| 11 | CDC 0092 | IMP | 25 | 25 | 25 | 50 |
| 12 | CDC 0103 | IMP | 25 | 25 | 25 | 25 |
| 13 | CDC 0439 | IMP | 25 | 25 | 25 | 25 |
| 14 | CDC 0241 | IMP | 25 | 12.5 | 25 | 25 |
| 15 | CDC 0508 | MDR | 50 | 50 | 25 | 25 |
| 16 | CDC 0353 | MDR | 25 | 25 | 25 | 25 |
| 17 | CDC 0357 | MDR | 25 | 25 | 25 | 25 |
| 18 | CDC 0246 | MDR | 50 | 12.5 | 25 | 50 |
| 19 | CDC 0250 | MDR | 25 | 25 | 25 | 25 |
| 20 | CDC 0064 | MDR | 25 | 25 | 25 | 25 |
| 21 | ATCC 27853 | QC strain | 25 | 25 | 25 | 25 |

AB-101 Lots 01, 02 and a purified extract of taspine for MIC are compared. The concentration of taspine at the highest concentration tested (i.e. 50%, relative to AB-101) is 10 μg/m L demonstrated for the first time from a bacteriologic perspective, taspine does not have activity as evaluated by this invitro test method against MSSA and MRSA. Taspine may have additional synergistic benefits to be included in the whole extract in the final product for the effective treatment for wound treating, bleeding treatment, and fighting infections. Results are shown in Table 12.

TABLE 12

| | | | | | | MIC (% | | | |
|---|---|---|---|---|---|---|---|---|---|
| | | | | | | relative | | | |
| | | | | | | to | | | |
| | | | | | | amount | | | |
| | | MIC (% vol./vol.) | | | | in AB-101) | | MIC (μg/mL) | |
| Strain ID | Characteristic | AB-101 Lot 01 | | AB-101 Lot 02 | | Taspine | | Methicillin | Mupirocin |
| CDC 218 | MupirocinR, MRSA | 12.5 | 12.5 | 12.5 | 12.5 | >50 | >50 | >64 | >256 |
| CDC 224 | MupirocinR, MRSA | 12.5 | 12.5 | 12.5 | 12.5 | >50 | >50 | >64 | >256 |
| CDC 228 | MupirocinR, MRSA | 3.13 | 3.13 | 3.13 | 3.13 | >50 | >50 | 64 | >256 |
| 1674606 | MupirocinR, MSSA | 50 | 50 | 50 | 50 | >50 | >50 | 2-8 | >256 |
| 1674607 | MupirocinR, MRSA | 12.5 | 12.5 | 12.5 | 12.5 | >50 | >50 | >64 | >256 |
| 1674608 | MupirocinR, MSSA | 12.5 | 12.5 | 25 | 50 | >50 | >50 | 2-4 | >256 |
| 1674611 | MupirocinR, MRSA | 6.25 | 6.25 | 12.5 | 12.5 | >50 | >50 | 64 | >256 |
| CDC 480 | MupirocinS, MRSA | 12.5 | 25 | 12.5 | 25 | >50 | >50 | 32-64 | ≤0.25 |
| CDC 481 | MupirocinS, MRSA | 12.5 | 12.5 | 12.5 | 12.5 | >50 | >50 | 16-32 | ≤0.25 |
| CDC 482 | MupirocinS, MRSA | 12.5 | 12.5 | 12.5 | 12.5 | >50 | >50 | 8-32 | ≤0.25 |
| CDC 483 | MupirocinS, MRSA | 12.5 | 12.5 | 12.5 | 12.5 | >50 | >50 | 64 | ≤0.25 |
| CDC 220 | MupirocinS, MRSA | 12.5 | 12.5 | 6.25 | 12.5 | >50 | >50 | 64->64 | ≤0.25 |
| CDC 227 | MupirocinS, MRSA | 6.25 | 6.25 | 12.5 | 12.5 | >50 | >50 | 64 | ≤0.25-0.5 |
| CDC 461 | MupirocinS, MSSA | 12.5 | 12.5 | 12.5 | 12.5 | >50 | >50 | 4-8 | ≤0.25-1 |
| CDC 462 | MupirocinS, MSSA | 6.25 | 12.5 | 6.25 | 12.5 | >50 | >50 | 8 | ≤0.25 |
| CDC 463 | MupirocinS, MSSA | 6.25 | 6.25 | 12.5 | 12.5 | >50 | >50 | 8-16 | ≤0.25-0.5 |
| CDC 464 | MupirocinS, MSSA | 12.5 | 12.5 | 12.5 | 12.5 | >50 | >50 | 2 | ≤0.25 |
| CDC 484 | MupirocinS, MSSA | 12.5 | 12.5 | 12.5 | 12.5 | >50 | >50 | 2 | ≤0.25 |
| CDC 485 | MupirocinS, MSSA | 50 | 50 | 50 | 50 | >50 | >50 | 2-4 | ≤0.25 |
| ATCC 29213 | QC control | 12.5 | 50-12.5 | 12.5 | 12.5 | >50 | >50 | 1-2 | ≤0.25-0.5 |

AB-101 Lots 01, 02 and a purified extract of taspine for MBC are compared. Demonstrated for the first time from a bacteriologic perspective, taspine does not have activity as evaluated by this invitro test method against MSSA and MRSA. Taspine may have additional synergistic benefits to be included in the whole extract in the final product for the effective treatment for wound treating, bleeding treatment, and fighting infections. Results are shown in Table 13.

TABLE 13

| | | | | | | MBC (% relative to amount in | |
|---|---|---|---|---|---|---|---|
| | | MBC (% vol./vol.) | | | | AB-101) | |
| Strain ID | Characteristic | AB-101 Lot 01 | | AB-101 Lot 02 | | Taspine | |
| CDC 218 | MupirocinR, MRSA | 50 | 50 | 50 | 50 | >50 | >50 |
| CDC 224 | MupirocinR, MRSA | 50 | 50 | 50 | 50 | >50 | >50 |
| CDC 228 | MupirocinR, MRSA | 6.25 | 6.25 | 6.25 | 6.25 | >50 | >50 |
| 1674606 | MupirocinR, MSSA | >50 | >50 | >50 | >50 | >50 | >50 |
| 1674607 | MupirocinR, MRSA | 12.5 | 12.5 | 25 | 25 | >50 | >50 |
| 1674608 | MupirocinR, MSSA | 50 | 50 | 50 | >50 | >50 | >50 |
| 1674611 | MupirocinR, MRSA | 12.5 | 12.5 | 50 | 50 | >50 | >50 |
| CDC 480 | MupirocinS, MRSA | 50 | 50 | 50 | 50 | >50 | >50 |
| CDC 481 | MupirocinS, MRSA | 50 | 50 | 50 | 50 | >50 | >50 |
| CDC 482 | MupirocinS, MRSA | 50 | 50 | 50 | 50 | >50 | >50 |
| CDC 483 | MupirocinS, MRSA | 50 | 50 | 50 | 50 | >50 | >50 |
| CDC 220 | MupirocinS, MRSA | 25 | 25 | 25 | 25 | >50 | >50 |
| CDC 227 | MupirocinS, MRSA | 12.5 | 12.5 | 25 | 25 | >50 | >50 |
| CDC 461 | MupirocinS, MSSA | 50 | 50 | 50 | 50 | >50 | >50 |
| CDC 462 | MupirocinS, MSSA | 12.5 | 50 | 50 | 50 | >50 | >50 |
| CDC 463 | MupirocinS, MSSA | 12.5 | 12.5 | 12.5 | 12.5 | >50 | >50 |
| CDC 464 | MupirocinS, MSSA | >50 | 50 | 50 | 50 | >50 | >50 |
| CDC 484 | MupirocinS, MSSA | 50 | 50 | 50 | 50 | >50 | >50 |
| CDC 485 | MupirocinS, MSSA | 50 | 50 | 50 | 50 | >50 | >50 |
| ATCC 29213 | QC control | 50-12.5 | 50-12.5 | 50 | 50 | >50 | >50 |

For treatment of topical injuries there are at least four types of bacteria pathogens that a topical drug needs to be effective against. They include Methicillin-susceptible Staphylococcus aureus (MSSA), Methicillin-resistant Staphylococcus aureus (MRSA), Pseudomonas aeruginosa and Streptococcus pyogenes. Pharmaceutical grade AB-101 is effective against all of these pathogens.

Tables 14 and 15 show an assay whose setup is same as what was used for screening S. aureus and P. aeruginosa (i.e. 2-fold microdilution). The 20 strains of S. pyogenes assayed were selected from the two rounds of antibiotic screening to identify Erythromycin and Tetracycline resistant strains. The only difference from previous AB-101 MIC/MBC assay setup is that the broth used is CAMHB supplemented with 2.5% laked horse blood, plating for MIC/MBC was on Tryptic Soy Agar (TSA) with 5% defibrinated sheep blood, and incubation was done at 37° C., 5% CO₂ overnight, as per Clinical and Laboratory Standards Institute recommendation for beta-hemolytic Streptococcus. For this feasibility study, AB-101 Lot 1 and Lot 2 was used. P. aeruginosa ATCC 27853 was tested as a positive control for AB-101 activity. For the first time AB-101 has demonstrated effectiveness against the S. aureus pathogen and effectiveness against Erythromycin and Tetracycline resistant strains.

TABLE 14
S. pyogenes AB-101 Lot 1 MIC/MBC Results

| Pathogen | | AB-101 Lot 1 | | | | MIC (μg/mL) | | | |
|---|---|---|---|---|---|---|---|---|---|
| Species | Strain ID | MIC (%) | | MBC (%) | | Erythromycin | Tetracycline | Mupirocin | Vancomycin |
| S. pyogenes | 1744264 | 6.25 | 6.25 | 6.25 | 6.25 | 2 | 0.25 | 0.25 | 0.5 |
| | 1744265 | 50 | 50 | >50 | 50 | >16 | 8 | 0.5 | 8 |
| | 1744271 | 6.25 | 6.25 | 6.25 | 6.25 | >16 | 16 | 0.5 | 0.5 |
| | 1744272 | 6.25 | 6.25 | 6.25 | 6.25 | >16 | 8 | 0.25 | 1 |
| | 1744275 | 3.125 | 3.125 | 3.125 | 6.25 | >16 | 0.25 | ≤0.125 | 0.25-0.5 |
| | 1744277 | 6.25 | 6.25 | 6.25 | 6.25 | 0.5 | 0.25 | ≤0.125 | 4 |
| | 1744285 | 3.125 | 3.125 | 3.125 | 3.125 | >16 | >16 | ≤0.125 | 0.5 |
| | 20658748 | 6.25 | 6.25 | 6.25 | 6.25 | 2 | 16 | ≤0.125 | 0.25-0.5 |
| | 20658749 | 3.125 | 3.125 | 3.125 | 3.125 | 1 | 16 | ≤0.125 | 0.5 |
| | 20658750 | 3.125 | 6.25 | 3.125 | 6.25 | 1 | >16 | ≤0.125 | 0.5 |
| | 2065754 | 3.125 | 6.25 | 3.125 | 6.25 | >16 | 16 | ≤0.125 | 0.5 |
| | 2065755 | 3.125 | 3.125 | 3.125 | 3.125 | 2 | >16 | ≤0.125 | 0.5 |
| | 2065756 | 3.125 | 6.25 | 3.125 | 6.25 | 2 | >16 | ≤0.125 | 0.5 |
| | 2065757 | 3.125 | 3.125 | 3.125 | 3.125 | >16 | 16 | ≤0.125 | 0.5 |
| | 2065759 | 3.125 | 3.125 | 3.125 | 3.125 | 4 | 16->16 | ≤0.125 | 0.5 |
| | 2065760 | 6.25 | 1.56 | 6.25 | 6.25 | 2 | >16 | ≤0.125 | 0.5 |
| | 2065761 | 3.125 | 3.125 | 3.125 | 6.25 | >16 | 16->16 | ≤0.125 | 0.5 |
| | 2065762 | 3.125 | 3.125 | 3.125 | 3.125 | 2 | >16 | ≤0.125 | 0.5 |
| | 2065763 | 3.125 | 3.125 | 3.125 | 6.25 | 2 | 16->16 | ≤0.125 | 0.5 |
| | 2065765 | 6.25 | 6.25 | 6.25 | 6.25 | >16 | 2-4 | ≤0.125 | 2-4 |
| P. | ATCC 27853 | 12.5 | 12.5 | 12.5 | 12.5 | | | | |
| aeruginosa | | | | | | | | | |

TABLE 15
S. pyogenes AB-101 Lot 1 MIC/MBC Results

| Pathogen | | AB-101 Lot 1 | | | | MIC (μg/mL) | | | |
|---|---|---|---|---|---|---|---|---|---|
| Species | Strain ID | MIC (%) | | MBC (%) | | Erythromycin | Tetracycline | Mupirocin | Vancomycin |
| S. pyogenes | 1744264 | 6.25 | 6.25 | 6.25 | 6.25 | 2 | 0.25 | 0.25 | 0.5 |
| | 1744265 | >50 | >50 | >50 | >50 | >16 | 8 | 0.5 | 8 |
| | 1744271 | 6.25 | 6.25 | 12.5 | 12.5 | >16 | 16 | 0.5 | 0.5 |
| | 1744272 | 6.25 | 6.25 | 6.25 | 6.25 | >16 | 8 | 0.25 | 1 |
| | 1744275 | 6.25 | 6.25 | 6.25 | 6.25 | >16 | 0.25 | ≤0.125 | 0.25-0.5 |
| | 1744277 | 6.25 | 6.25 | 6.25 | 6.25 | 0.5 | 0.25 | ≤0.125 | 4 |
| | 1744285 | 6.25 | 6.25 | 6.25 | 6.25 | >16 | >16 | ≤0.125 | 0.5 |
| | 20658748 | 6.25 | 6.25 | 6.25 | 6.25 | 2 | 16 | ≤0.125 | 0.25-0.5 |
| | 20658749 | 6.25 | 6.25 | 6.25 | 6.25 | 1 | 16 | ≤0.125 | 0.5 |
| | 20658750 | 6.25 | 6.25 | 6.25 | 6.25 | 1 | >16 | ≤0.125 | 0.5 |
| | 2065754 | 6.25 | 6.25 | 6.25 | 6.25 | >16 | 16 | ≤0.125 | 0.5 |
| | 2065755 | 6.25 | 6.25 | 6.25 | 6.25 | 2 | >16 | ≤0.125 | 0.5 |
| | 2065756 | 6.25 | 6.25 | 6.25 | 6.25 | 2 | >16 | ≤0.125 | 0.5 |
| | 2065757 | 6.25 | 6.25 | 6.25 | 6.25 | >16 | 16 | ≤0.125 | 0.5 |
| | 2065759 | 6.25 | 6.25 | 6.25 | 6.25 | 4 | 16->16 | ≤0.125 | 0.5 |
| | 2065760 | 6.25 | 6.25 | 6.25 | 6.25 | 2 | >16 | ≤0.125 | 0.5 |
| | 2065761 | 6.25 | 6.25 | 6.25 | 6.25 | >16 | 16->16 | ≤0.125 | 0.5 |
| | 2065762 | 6.25 | 6.25 | 6.25 | 6.25 | 2 | >16 | ≤0.125 | 0.5 |
| | 2065763 | 6.25 | 6.25 | 6.25 | 6.25 | 2 | 16->16 | ≤0.125 | 0.5 |
| | 2065765 | 6.25 | 6.25 | 6.25 | 6.25 | >16 | 2-4 | ≤0.125 | 2-4 |
| P. | ATCC 27853 | 12.5 | 12.5 | 25 | 25 | | | | |
| aeruginosa | | | | | | | | | |

### Example 4—Clinical Usage of Pharmaceutical grade AB-101

Pharmaceutical grade AB-101 show effectiveness in stopping bleeding, coagulating blood, forming a protective film and preventing infection in 3 different case studies. The protocol for all of the studies was to wash the wound, apply enough drop of AB-101 to rub in and around the wound and continue this practice twice daily for 7 days.

Case 1: AB-101 treats a wound on the thigh area of the upper left leg. AB-101 is applied to a bleeding clean wound puncture. AB-101 is immediately rubbed on top of the wound. The wound immediately stops bleeding and a film is formed across the wound. Stoppage of bleeding is curtailed across the early bleed period and promoting early blood clotting. The AB-101 protective film stays in place and does not dissolve. At day 3 the wound continues to heal and decreased in size. After the 7-day treatment period, following application of AB-101 twice a day, the patient self-assessed the wound and determined it had decreased in size by 50%. During this period, the bleeding of the wound was ceased, and did not have any bleeding breakout periods. The wound remained protected by the AB-101 film and no infection occurred.

Case 2: In a separate case, a wound occurred to the inside left thigh. AB-101 is applied to a bleeding clean wound puncture. AB-101 is immediately rubbed on top of the wound. The wound immediately stops bleeding and a film is formed across the wound. Immediately after application of AB-101 the wound stopped bleeding and the AB-101 film formed over the wound. As in Case 1, AB-101 caused the blood to immediately to coagulate and promoted early blood clotting. During the 7-day period, no breakout bleeding occurred. Following the AB-101 application protocol, AB-101 was self-assessed to decrease the wound size by 40%. The wound remained protected by the AB-101 film throughout the application period and no infection occurred.

Case 3: In a separate case, a wound occurred to the top of the right foot. AB-101 is applied to a bleeding clean wound puncture. AB-101 is immediately rubbed on top of the wound. The wound immediately stops bleeding and a film is formed across the wound. This example was shown to demonstrate the immediate wound blood clotting capability of AB-101 to be able to stop the bleeding and form a film on the wound.

1. A method of treating a condition in a subject comprising contacting the subject in need thereof with a treated bandage comprising a therapeutically effective amount of filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, at least about 0.1 PPM of Dimethylcedrusin.

2. A method of treating a condition in a subject comprising contacting the subject in need thereof with a treated bandage comprising a pharmaceutical composition containing filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, at least about 0.1 PPM of Dimethylcedrusin.

3. The method of claim 1, wherein the condition is selected from the group consisting of treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

4. The method of claim 2, wherein the condition is selected from the group consisting of treatment of wounds in a subject, treatment of chronic wounds in a subject, treatment of traumatic wounds in a subject, treatment of acute wounds in a subject, treating or preventing or reducing the risk of a bacterial infection of wounds in a subject, inducing blood clotting of a wound in a subject, promotion of blood coagulation in a subject, promotion of clot formation in a subject, promotion of blood clotting by accelerating the normal platelet clotting pathway in a subject, promotion of wound healing in a subject, promotion of wound occlusion in a subject, promotion of wound occlusion thereby reducing scarring in a subject, promotion of tissue regeneration in a subject, promotion of tissue growth in a subject, promotion of tissue growth thereby accelerating wound healing in a subject, or a combination thereof.

5. The method of claim 1, wherein the therapeutically effective amount of filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, at least about 0.1 PPM of Dimethylcedrusin, is first applied to a bandage to form a treated bandage.

6. The method of claim 1, wherein the therapeutically effective amount of filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, at least about 0.1 PPM of Dimethylcedrusin, is first impregnated into a bandage to form a treated bandage.

7. The method of claim 2, wherein the pharmaceutical composition containing filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, at least about 0.1 PPM of Dimethylcedrusin, is first applied to a bandage to form a treated bandage.

8. The method of claim 2, wherein the pharmaceutical composition containing filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, at least about 0.1 PPM of Dimethylcedrusin, is first impregnated into a bandage to form a treated bandage.
